SEQUENCE LISTING (78 sequences)

SEQ ID NO: 1 - DNA, 1677 bp, Pseudomonas chlororaphis
atgagtaacaagaataacgatgacctgaaacgccaagcctcggaaaacactctgggcctgaatcctgtcatcggtttacgtcggaaagatctgcttacctctgctcgtatggtgctgacccaggccatcaaacaaccgctgcacagcgccaaacatgtggcccatttcggcgtcgaactgaagaacgtgatgttcggcaaatcgacgctgcaaccggagagcgatgaccgtcgcttcaacgatccggcctggagccagaacccgctgtacaagcgttacctgcaaacctacctggcgtggcgcaaggagcttcacgactggatcggccacagcaacctctccgaacaggacatcagtcgcgcccacttcgtcatcaacctgatgaccgaagccatggcgccgaccaacaccgccgccaaccccgcggcggtcaaacgcttcttcgagaccggcggcaaaagcctgctcgatggcctctcccatctggccaaggacctggtgcacaacggcggcatgccgagccaggtcaacatggacgccttcgagatcggcaagaacctgggcaccagtgaaggcgcggtggtatttcgcaacgacgtgctggaactgatccagtaccgcccggtgaccgagcaggtgcacgaacgcccgctgctggtggtcccgccacagatcaacaagttctacgtcttcgacctcagcccggacaagagcctggcgcgtttctgcctgcgcaacggcgtgcagacctttatcgtcagttggcgcaacccgaccaaggcgcagcgcgagtggggcctgtcgacctacatcgaagcgctgaaagaagcggtcgacgtggtcaccgccatcaccggcagcaaggacgtgaacatgctcggcgcctgctccggtggcatcacctgcaccgcgctgctggggcactacgcggccctgggcgagaagaaggtcaacgccctgaccctgctggtcagcgtgctcgataccaccctcgacacccaggtggcgctgttcgtcgacgagcagaccctggaagccgccaagcgtcattcctatcaggctggcgtgctggaaggccgcgacatggccaaggtcttcgcctggatgcggcccaacgacctgatctggaactactgggtgaacaactacctgctgggcaacgagccgccggtgttcgacatcctgttctggaacaacgacaccacccggttgcccgcggccttccacggcgacctgatcgaaatgttcaaaaacaatccactgatccgccccaacgcactggaagtgtgcggtacgccgatcgacctgaaacaggtcaccgccgacatcttctctctggccggcaccaacgatcacatcaccccatggaagtcctgctacaagtcggcccaactgttcggcggcaaggtcgaattcgtcctctccagcagcgggcatatccagagcatcctcaacccgccgggcaaccccaagtcgcgctacatgaccagcgaagagatgccgcccagcgccgacgactggcaggaaaactccaccaagcacactgactcctggtggctgcactggcaggcctggcaggcggagcgctcgggcaaactgaaaaagacgccgagcattctcggcaacaaggcgtatccagcggccgaggcggcgccgggtacttatgtccatgaacgt

SEQ ID NO: 2 - protein, 559 aa, Pseudomonas chlororaphis
MSNKNNDDLKRQASENTLGLNPVIGLRRKDLLTSARMVLTQAIKQPLHSAKHVAHFGVELKNVMFGKSTLQPESDDRRFNDPAWSQNPLYKRYLQTYLAWRKELHDWIGHSNLSEQDISRAHFVINLMTEAMAPTNTAANPAAVKRFFETGGKSLLDGLSHLAKDLVHNGGMPSQVNMDAFEIGKNLGTSEGAVVFRNDVLELIQYRPVTEQVHERPLLVVPPQINKFYVFDLSPDKSLARFCLRNGVQTFIVSWRNPTKAQREWGLSTYIEALKEAVDVVTAITGSKDVNMLGACSGGITCTALLGHYAALGEKKVNALTLLVSVLDTTLDTQVALFVDEQTLEAAKRHSYQAGVLEGRDMAKVFAWMRPNDLIWNYWVNNYLLGNEPPVFDILFWNNDTTRLPAAFHGDLIEMFKNNPLIRPNALEVCGTPIDLKQVTADIFSLAGTNDHITPWKSCYKSAQLFGGKVEFVLSSSGHIQSILNPPGNPKSRYMTSEEMPPSADDWQENSTKHTDSWWLHWQAWQAERSGKLKKTPSILGNKAYPAAEAAPGTYVHER

SEQ ID NO: 3 - DNA, 1677 bp, Pseudomonas putida
atgagtaacaagaacaacgatgagctacagcggcaggcctcggaaaacaccctggggctgaacccggtcatcggcatccgccgcaaggacctgttgagctcggcacgcaccgtgctgcgccaggccgtgcgccaaccgctgcacagcgccaagcatgtggctcactttggcctggagctgaagaacgtgttgctgggcaaatccagcctggccccggacagcgacgaccgtcgcttcaatgacccggcctggagcaacaacccgctgtaccgccgctacctgcaaacctacctggcctggcgcaaggagctgcaggactgggtgagcagcagcgacctgtccccccaggacatcagccgcggccagttcgtcatcaacctgatgaccgaggccatggcgccgaccaataccctgtccaacccggctgcggtcaaacgcttcttcgaaaccggcggcaagagcctgctcgatggcctgtccaacctggccaaggacatggtcaacaacggcggcatgcccagccaggtgaacatggatgccttcgaagtgggcaagaacctgggcaccagcgaaggcgcggtggtgtaccgcaacgatgtgctggaactgatccagtacagccccatcaccgagcaggtgcatgcccgtccgctgctggtggtgccaccgcagatcaacaagttctacgtgttcgacctcagcccggaaaagagcctggcgcgcttctgcctgcgctcgcagcagcagaccttcatcatcagctggcgcaacccgaccaaggcccagcgtgaatggggcctgtccacctacatcgatgcgctgaaagaagccgtcgacgcggtgctgtcgattaccggcagcaaggacctgaacatgctcggcgcctgctccggtggcatcacttgtaccgcactggtgggccactatgccgccattggcgagaacaaggtcaacgccctgaccctgctggtcagcgtgctggacaccaccatggacaaccaggttgctttgtttgtcgacgagcagaccttggaggccgccaagcgccactcctatcaggcgggcgtgctggaaggcagcgaaatggccaaggtgttcgcctggatgcgccccaacgacctgatctggaactactgggtaaacaactacctgctcggcaatgagccccccgtgttcgacatcctgttctggaacaacgacaccacgcgcctgccggccgccttccacggcgacctgatcgaaatgttcaagagcaacccgctgacccgccccgacgccctggaagtgtgcggcaccgcgatcgacctgaaacaggtcaaatgcgacatctacagcctcgccggcaccaacgaccacatcaccccctggccgtcatgctaccgctcggcacatctgttcggcggcaagatcgaattcgtactgtccaacagcgggcatatccagagcatcctcaacccgccgggcaacccgaaggcacgtttcatgaccggtgccgatcgcccgggtgacccggtggcctggcaggaaaatgccatcaagcatgcagactcctggtggttgcactggcagagttggctgggcgagcgtgccggcgcgctgaaaaaggcaccgacccgcctgggcaaccgtacctatgccgccggcgaagcctccccaggcacctacgttcacgagcgt

SEQ ID NO: 4 - protein, 559 aa, Pseudomonas putida
MSNKNNDELQRQASENTLGLNPVIGIRRKDLLSSARTVLRQAVRQPLHSAKHVAHFGLELKNVLLGKSSLAPDSDDRRFNDPAWSNNPLYRRYLQTYLAWRKELQDWVSSSDLSPQDISRGQFVINLMTEAMAPTNTLSNPAAVKRFFETGGKSLLDGLSNLAKDMVNNGGMPSQVNMDAFEVGKNLGTSEGAVVYRNDVLELIQYSPITEQVHARPLLVVPPQINKFYVFDLSPEKSLARFCLRSQQQTFIISWRNPTKAQREWGLSTYIDALKEAVDAVLSITGSKDLNMLGACSGGITCTALVGHYAAIGENKVNALTLLVSVLDTTMDNQVALFVDEQTLEAAKRHSYQAGVLEGSEMAKVFAWMRPNDLIWNYWVNNYLLGNEPPVFDILFWNNDTTRLPAAFHGDLIEMFKSNPLTRPDALEVCGTAIDLKQVKCDIYSLAGTNDHITPWPSCYRSAHLFGGKIEFVLSNSGHIQSILNPPGNPKARFMTGADRPGDPVAWQENAIKHADSWWLHWQSWLGERAGALKKAPTRLGNRTYAAGEASPGTYVHER

SEQ ID NO: 5 - DNA, 1677 bp, Pseudomonas resinovorans
atgagcaacaagaacaatgaagacctgcagcgccaggcctcggacaacaccctgaacctcaatccggtgataggtatccgcggcaaggatctgctgtcttcagcacggatggtgctgttgcaagccatcaagcaacctttccacagtgcaaaacacgtcgcccatttcggcctggaattgaaaaacgtgttgcttggccagtccggtctgcaaccggaagccgatgaccgtcgcttcaatgatccggcctggagccagaacccgctgtacaagcgctatctgcagacctacctcgcctggcgcaaggagctgcacagctggatcgacgagagcaacctctcgtcacaggatgccagtcgcggccacttcgtcatcaacctgatgaccgaggcgatggcgcccaccaacagcatggccaacccggccgcagtcaaacggttcttcgagaccggcggcaagagcctgctggatggcctctcccacctggccaaggacatggtgaacaacggcggcatgccgagccaggtcaacatggatgccttcgaggtcggccagaacctcgccacgaccgaaggtgcggtggttttccgcaatgacgtgctggaactgatccagtacaagcccatcaccgagagcgtgtacgaacgcccgctactcgtggtgccgccccagatcaacaagttctacgtcttcgacctgtcaccggagaagagcctcgcgcgcttctgcctgcgcagcaacctgcagaccttcatcgtcagctggcgcaacccgaccaaggcgcagcgcgagtggggcctgtccacgtacatcgaggccctcaaggaggccattgacgtcatcctgaagatcaccggcgccaaggacctcaacatcctcggcgcgtgctccggcggcatcaccacggtcgccctgctcggccactaccaggccattggtgaaaccaaggtcaatgccttcacccagatggtcagcgtgctcgacttcaacctcgatagccaggtcgcgctgttcgccgacgagcagaccctcgaagccgccaagcgccgctcgtaccaggccggcgtactggaaggcaaagacatggccaaggtcttcgcctggatgcgccccaacgacctgatctggaactactgggtcaacaactacctgctgggcaacgaaccgccggccttcgacatcctctattggaacaacgacaccacgcgtctgcctgcagcgttccatggcgaactggtggaaatgttcaagaccaatgccctgacccgtccaaatgccctggaggtctgtggcacgcccatcgacctgaagcaagtcaccagcgacttcttctgccttgccggcaccaccgaccacattaccccctgggaagcctgctaccgctcggcgctgctgctgggcggcaaatgcgagttcgtgctgtccaacagcggccacatccagagcattctcaacccaccgggcaaccccaaggcacgcttctccaccggcagcgagatgccgaaagatccgaaggcctggctggaaaatgcgaccaagcacgccgactcctggtggctgcattggcagcagtggatcggcgaacgctccggcaagaccaagaaagccagctttaccctgggcaacaaggccttcccggccggtgaagcctcgcccggcacctatgtgcacgagcga

SEQ ID NO: 6 - protein, 559 aa, Pseudomonas resinovorans
MSNKNNEDLQRQASDNTLNLNPVIGIRGKDLLSSARMVLLQAIKQPFHSAKHVAHFGLELKNVLLGQSGLQPEADDRRFNDPAWSQNPLYKRYLQTYLAWRKELHSWIDESNLSSQDASRGHFVINLMTEAMAPTNSMANPAAVKRFFETGGKSLLDGLSHLAKDMVNNGGMPSQVNMDAFEVGQNLATTEGAVVFRNDVLELIQYKPITESVYERPLLVVPPQINKFYVFDLSPEKSLARFCLRSNLQTFIVSWRNPTKAQREWGLSTYIEALKEAIDVILKITGAKDLNILGACSGGITTVALLGHYQAIGETKVNAFTQMVSVLDFNLDSQVALFADEQTLEAAKRRSYQAGVLEGKDMAKVFAWMRPNDLIWNYWVNNYLLGNEPPAFDILYWNNDTTRLPAAFHGELVEMFKTNALTRPNALEVCGTPIDLKQVTSDFFCLAGTTDHITPWEACYRSALLLGGKCEFVLSNSGHIQSILNPPGNPKARFSTGSEMPKDPKAWLENATKHADSWWLHWQQWIGERSGKTKKASFTLGNKAFPAGEASPGTYVHER

SEQ ID NO: 7 - DNA, 1677 bp, Pseudomonas aeruginosa
atgagtcagaagaacaataacgagcttcccaagcaagccgcggaaaacacgctgaacctgaatccggtgatcggcatccggggcaaggacctgctcacctccgcgcgcatggtcctgctccaggcggtgcgccagccgctgcacagcgccaggcacgtggcgcatttcagcctggagctgaagaacgtcctgctcggccagtcggagctacgcccaggcgatgacgaccgacgcttttccgatccggcctggagccagaatccactgtacaagcgctacatgcagacctacctggcctggcgcaaggagctgcacagctggatcagccacagcgacctgtcgccgcaggacatcagtcgtggccagttcgtcatcaacctgctgaccgaggcgatgtcgccgaccaacagcctgagcaacccggcggcggtcaagcgcttcttcgagaccggcggcaagagcctgctggacggcctcggccacctggccaaggacctggtgaacaacggcgggatgccgagccaggtggacatggacgccttcgaggtgggcaagaacctggccaccaccgagggcgccgtggtgttccgcaacgacgtgctggaactgatccagtaccggccgatcaccgagtcggtgcacgaacgcccgctgctggtggtgccgccgcagatcaacaagttctacgtcttcgacctgtcgccggacaagagcctggcgcgcttctgcctgcgcaacggcgtgcagaccttcatcgtcagttggcgcaacccgaccaagtcgcagcgcgaatggggcctgaccacctatatcgaggcgctcaaggaggccatcgaggtagtcctgtcgatcaccggcagcaaggacctcaacctcctcggcgcctgctccggcgggatcaccaccgcgaccctggtcggccactacgtggccagcggcgagaagaaggtcaacgccttcacccaactggtcagcgtgctcgacttcgaactgaatacccaggtcgcgctgttcgccgacgagaagactctggaggccgccaagcgtcgttcctaccagtccggcgtgctggagggcaaggacatggccaaggtgttcgcctggatgcgccccaacgacctgatctggaactactgggtcaacaactacctgctcggcaaccagccgccggcgttcgacatcctctactggaacaacgacaccacgcgcctgcccgccgcgctgcacggcgagttcgtcgaactgttcaagagcaacccgctgaaccgccccggcgccctggaggtctccggcacgcccatcgacctgaagcaggtgacttgcgacttctactgtgtcgccggtctgaacgaccacatcaccccctgggagtcgtgctacaagtcggccaggctgctgggtggcaagtgcgagttcatcctctccaacagcggtcacatccagagcatcctcaacccaccgggcaaccccaaggcacgcttcatgaccaatccggaactgcccgccgagcccaaggcctggctggaacaggccggcaagcacgccgactcgtggtggttgcactggcagcaatggctggccgaacgctccggcaagacccgcaaggcgcccgccagcctgggcaacaagacctatccggccggcgaagccgcgcccggaacctacgtgcatgaacga

SEQ ID NO: 8 - protein, 559 aa, Pseudomonas aeruginosa
MSQKNNNELPKQAAENTLNLNPVIGIRGKDLLTSARMVLLQAVRQPLHSARHVAHFSLELKNVLLGQSELRPGDDDRRFSDPAWSQNPLYKRYMQTYLAWRKELHSWISHSDLSPQDISRGQFVINLLTEAMSPTNSLSNPAAVKRFFETGGKSLLDGLGHLAKDLVNNGGMPSQVDMDAFEVGKNLATTEGAVVFRNDVLELIQYRPITESVHERPLLVVPPQINKFYVFDLSPDKSLARFCLRNGVQTFIVSWRNPTKSQREWGLTTYIEALKEAIEVVLSITGSKDLNLLGACSGGITTATLVGHYVASGEKKVNAFTQLVSVLDFELNTQVALFADEKTLEAAKRRSYQSGVLEGKDMAKVFAWMRPNDLIWNYWVNNYLLGNQPPAFDILYWNNDTTRLPAALHGEFVELFKSNPLNRPGALEVSGTPIDLKQVTCDFYCVAGLNDHITPWESCYKSARLLGGKCEFILSNSGHIQSILNPPGNPKARFMTNPELPAEPKAWLEQAGKHADSWWLHWQQWLAERSGKTRKAPASLGNKTYPAGEAAPGTYVHER

SEQ ID NO: 9 - DNA, 26 bp, Artificial Sequence
  primer for recognition site insertion
gcaatgcccggagccgggctagctag

SEQ ID NO: 10 - DNA, 42 bp, Artificial Sequence
  primer for recognition site insertion
gtcatcgttattcttgttactcatgatttgattgtctctctg

SEQ ID NO: 11 - DNA, 42 bp, Artificial Sequence
  primer for PHA synthase cloning
cagagagacaatcaaatcatgagtaacaagaataacgatgac

SEQ ID NO: 12 - DNA, 39 bp, Artificial Sequence
  primer for PHA synthase cloning
gcactcatgcaagcgttaacgttcatggacataagtacc

SEQ ID NO: 13 - DNA, 39 bp, Artificial Sequence
  primer for recognition site insertion
ggtacttatgtccatgaacgttaacgcttgcatgagtgc

SEQ ID NO: 14 - DNA, 42 bp, Artificial Sequence
  primer for recognition site insertion
ctcatcgttgttcttgttactcatgatttgattgtctctctg

SEQ ID NO: 15 - DNA, 42 bp, Artificial Sequence
  primer for PHA synthase cloning
cagagagacaatcaaatcatgagtaacaagaacaacgatgag

SEQ ID NO: 16 - DNA, 37 bp, Artificial Sequence
  primer for PHA synthase cloning
gcactcatgcaagcgtcaacgctcgtgaacgtaggtg

SEQ ID NO: 17 - DNA, 37 bp, Artificial Sequence
  primer for recognition site insertion
cacctacgttcacgagcgttgacgcttgcatgagtgc

SEQ ID NO: 18 - DNA, 42 bp, Artificial Sequence
  primer for recognition site insertion
gtcttcattgttcttgttgctcatgatttgattgtctctctg

SEQ ID NO: 19 - DNA, 42 bp, Artificial Sequence
  primer for PHA synthase cloning
cagagagacaatcaaatcatgagcaacaagaacaatgaagac

SEQ ID NO: 20 - DNA, 37 bp, Artificial Sequence
  primer for PHA synthase cloning
gcactcatgcaagcgtcatcgctcgtgcacataggtg

SEQ ID NO: 21 - DNA, 37 bp, Artificial Sequence
  primer for recognition site insertion
cacctatgtgcacgagcgatgacgcttgcatgagtgc

SEQ ID NO: 22 - DNA, 42 bp, Artificial Sequence
  primer for recognition site insertion
ctcgttattgttcttctgactcatgatttgattgtctctctg

SEQ ID NO: 23 - DNA, 42 bp, Artificial Sequence
  primer for PHA synthase cloning
cagagagacaatcaaatcatgagtcagaagaacaataacgag

SEQ ID NO: 24 - DNA, 38 bp, Artificial Sequence
  primer for PHA synthase cloning
gcactcatgcaagcgtcatcgttcatgcacgtaggttc

SEQ ID NO: 25 - DNA, 38 bp, Artificial Sequence
  primer for recognition site insertion
gaacctacgtgcatgaacgatgacgcttgcatgagtgc

SEQ ID NO: 26 - DNA, 22 bp, Artificial Sequence
  primer for recognition site insertion
gaaattgttatccgcctgcagg

SEQ ID NO: 27 - DNA, 33 bp, Artificial Sequence
  primer for E130D mutation in PhaC1Ps6-19
atcaacctcatgaccgatgcgatggcgccgacc

SEQ ID NO: 28 - DNA, 33 bp, Artificial Sequence
  primer for E130D mutation in PhaC1Ps6-19
ggtcggcgccatcgcatcggtcatgaggttgat

SEQ ID NO: 29 - DNA, 33 bp, Artificial Sequence
  primer for S325T mutation in PhaC1Ps6-19
ctgaccttgctggtgaccgtgcttgataccacc

SEQ ID NO: 30 - DNA, 33 bp, Artificial Sequence
  primer for S325T mutation in PhaC1Ps6-19
ggtggtatcaagcacggtcaccagcaaggtcag

SEQ ID NO: 31 - DNA, 30 bp, Artificial Sequence
  primer for S477G mutation in PhaC1Ps6-19
gaattcgtgctgtcgagcggcgggcatatc

SEQ ID NO: 32 - DNA, 30 bp, Artificial Sequence
  primer for S477G mutation in PhaC1Ps6-19
gatatgcccgccgctcgacagcacgaattc

SEQ ID NO: 33 - DNA, 28 bp, Artificial Sequence
  primer for Q481K mutation in PhaC1Ps6-19
gggcatatcaaaagcatcctgaacccgc

SEQ ID NO: 34 - DNA, 28 bp, Artificial Sequence
  primer for Q481K mutation in PhaC1Ps6-19
gcgggttcaggatgcttttgatatgccc

SEQ ID NO: 35 - DNA, 33 bp, Artificial Sequence
  primer for E130D mutation in PhaC1Pch
atcaacctgatgaccgatgccatggcgccgacc

SEQ ID NO: 36 - DNA, 33 bp, Artificial Sequence
  primer for E130D mutation in PhaC1Pch
ggtcggcgccatggcatcggtcatcaggttgat

SEQ ID NO: 37 - DNA, 28 bp, Artificial Sequence
  primer for Q481K mutation in PhaC1Pch
gggcatatcaaaagcatcctcaacccgc

SEQ ID NO: 38 - DNA, 28 bp, Artificial Sequence
  primer for Q481K mutation in PhaC1Pch
gcgggttgaggatgcttttgatatgccc

SEQ ID NO: 39 - DNA, 30 bp, Artificial Sequence
  primer for S477F mutation in PhaC1Pch
gaattcgtcctctccagctttgggcatatc

SEQ ID NO: 40 - DNA, 30 bp, Artificial Sequence
  primer for S477F mutation in PhaC1Pch
gatatgcccaaagctggagaggacgaattc

SEQ ID NO: 41 - DNA, 33 bp, Artificial Sequence
  primer for S325T mutation in PhaC1Pch
ctgaccctgctggtcaccgtgctcgataccacc

SEQ ID NO: 42 - DNA, 33 bp, Artificial Sequence
  primer for S325T mutation in PhaC1Pch
ggtggtatcgagcacggtgaccagcagggtcag

SEQ ID NO: 43 - DNA, 30 bp, Artificial Sequence
  primer for S477G mutation in PhaC1Pch
gaattcgtcctctccagcggcgggcatatc

SEQ ID NO: 44 - DNA, 30 bp, Artificial Sequence
  primer for S477G mutation in PhaC1Pch
gatatgcccgccgctggagaggacgaattc

SEQ ID NO: 45 - DNA, 33 bp, Artificial Sequence; identical to SEQ ID NO: 35
  primer for E130D mutation in PhaC1Ppu

SEQ ID NO: 46 - DNA, 33 bp, Artificial Sequence; identical to SEQ ID NO: 36
  primer for E130D mutation in PhaC1Ppu

SEQ ID NO: 47 - DNA, 28 bp, Artificial Sequence
  primer for Q481K mutation in PhaC1Ppu
gggcatatcaaaagcatcctcaacccgc

SEQ ID NO: 48 - DNA, 28 bp, Artificial Sequence
  primer for Q481K mutation in PhaC1Ppu
gcgggttgaggatgcttttgatatgccc

SEQ ID NO: 49 - DNA, 30 bp, Artificial Sequence
  primer for S477F mutation in PhaC1Ppu
gaattcgtactgtccaactttgggcatatc

SEQ ID NO: 50 - DNA, 30 bp, Artificial Sequence
  primer for S477F mutation in PhaC1Ppu
gatatgcccaaagttggacagtacgaattc

SEQ ID NO: 51 - DNA, 33 bp, Artificial Sequence
  primer for S325T mutation in PhaC1Ppu
ctgaccctgctggtcaccgtgctggacaccacc

SEQ ID NO: 52 - DNA, 33 bp, Artificial Sequence
  primer for S325T mutation in PhaC1Ppu
ggtggtgtccagcacggtgaccagcagggtcag

SEQ ID NO: 53 - DNA, 30 bp, Artificial Sequence
  primer for S477G mutation in PhaC1Ppu
gaattcgtactgtccaacggcgggcatatc

SEQ ID NO: 54 - DNA, 30 bp, Artificial Sequence
  primer for S477G mutation in PhaC1Ppu
gatatgcccgccgttggacagtacgaattc

SEQ ID NO: 55 - DNA, 33 bp, Artificial Sequence
  primer for E130D mutation in PhaC1Pre
atcaacctgatgaccgatgcgatggcgcccacc

SEQ ID NO: 56 - DNA, 33 bp, Artificial Sequence
  primer for E130D mutation in PhaC1Pre
ggtgggcgccatcgcatcggtcatcaggttgat

SEQ ID NO: 57 - DNA, 28 bp, Artificial Sequence
  primer for Q481K mutation in PhaC1Pre
ggccacatcaaaagcattctcaacccac

SEQ ID NO: 58 - DNA, 28 bp, Artificial Sequence
  primer for Q481K mutation in PhaC1Pre
gtgggttgagaatgcttttgatgtggcc

SEQ ID NO: 59 - DNA, 30 bp, Artificial Sequence
  primer for S477F mutation in PhaC1Pre
gagttcgtgctgtccaactttggccacatc

SEQ ID NO: 60 - DNA, 30 bp, Artificial Sequence
  primer for S477F mutation in PhaC1Pre
gatgtggccaaagttggacagcacgaactc

SEQ ID NO: 61 - DNA, 33 bp, Artificial Sequence
  primer for S325T mutation in PhaC1Pre
ttcacccagatggtcaccgtgctcgacttcaac

SEQ ID NO: 62 - DNA, 33 bp, Artificial Sequence
  primer for S325T mutation in PhaC1Pre
gttgaagtcgagcacggtgaccatctgggtgaa

SEQ ID NO: 63 - DNA, 30 bp, Artificial Sequence
  primer for S477G mutation in PhaC1Pre
gagttcgtgctgtccaacggcggccacatc

SEQ ID NO: 64 - DNA, 30 bp, Artificial Sequence
  primer for S477G mutation in PhaC1Pre
gatgtggccgccgttggacagcacgaactc

SEQ ID NO: 65 - DNA, 33 bp, Artificial Sequence
  primer for E130D mutation in PhaC1Pae
atcaacctgctgaccgatgcgatgtcgccgacc

SEQ ID NO: 66 - DNA, 33 bp, Artificial Sequence
  primer for E130D mutation in PhaC1Pae
ggtcggcgacatcgcatcggtcagcaggttgat

SEQ ID NO: 67 - DNA, 28 bp, Artificial Sequence
  primer for Q481K mutation in PhaC1Pae
ggtcacatcaaaagcatcctcaacccac

SEQ ID NO: 68 - DNA, 28 bp, Artificial Sequence
  primer for Q481K mutation in PhaC1Pae
gtgggttgaggatgcttttgatgtgacc

SEQ ID NO: 69 - DNA, 30 bp, Artificial Sequence
  primer for S477F mutation in PhaC1Pae
gagttcatcctctccaactttggtcacatc

SEQ ID NO: 70 - DNA, 30 bp, Artificial Sequence
  primer for S477F mutation in PhaC1Pae
gatgtgaccaaagttggagaggatgaactc

SEQ ID NO: 71 - DNA, 33 bp, Artificial Sequence
  primer for S325T mutation in PhaC1Pae
ttcacccaactggtcaccgtgctcgacttcgaa

SEQ ID NO: 72 - DNA, 33 bp, Artificial Sequence
  primer for S325T mutation in PhaC1Pae
ttcgaagtcgagcacggtgaccagttgggtgaa

SEQ ID NO: 73 - DNA, 30 bp, Artificial Sequence
  primer for S477G mutation in PhaC1Pae
gagttcatcctctccaacggcggtcacatc

SEQ ID NO: 74 - DNA, 30 bp, Artificial Sequence
  primer for S477G mutation in PhaC1Pae
gatgtgaccgccgttggagaggatgaactc

SEQ ID NO: 75 - DNA, 1677 bp, Pseudomonas sp.
atgagtaacaagagtaacgatgagttgaagtatcaagcctctgaaaacaccttggggcttaatcctgtcgttgggctgcgtggaaaggatctactggcttctgctcgaatggtgcttaggcaggccatcaagcaaccggtgcacagcgtcaaacatgtcgcgcactttggtcttgaactcaagaacgtactgctgggtaaatccgggctgcaaccgaccagcgatgaccgtcgcttcgccgatccggcctggagccagaacccgctctataaacgttatttgcaaacctacctggcgtggcgcaaggaactccacgactggatcgatgaaagtaacctcgcccccaaggatgtggcgcgtgggcacttcgtgatcaacctcatgaccgaagcgatggcgccgaccaacaccgcggccaacccggcggcagtcaaacgcttttttgaaaccggtggcaaaagcctgctcgacggcctctcgcacctggccaaggatctggtacacaacggcggcatgccgagccaggtcaacatgggtgcattcgaggtcggcaagagcctgggcgtgaccgaaggcgcggtggtgtttcgcaacgatgtgctggaactgatccagtacaagccgaccaccgagcaggtatacgaacgcccgctgctggtggtgccgccgcagatcaacaagttctacgttttcgacctgagcccggacaagagcctggcgcggttctgcctgcgcaacaacgtgcaaacgttcatcgtcagctggcgaaatcccaccaaggaacagcgagagtggggcctgtcgacctacatcgaagccctcaaggaagcggttgacgtcgttaccgcgatcaccggcagcaaagacgtgaacatgctcggggcctgctccggcggcatcacttgcactgcgctgctgggccattacgcggcgattggcgaaaacaaggtcaacgccctgaccttgctggtgagcgtgcttgataccaccctcgacagcgacgtcgccctgttcgtcaatgaacagacccttgaagccgccaagcgccactcgtaccaggccggcgtactggaaggccgcgacatggcgaaggtcttcgcctggatgcgccccaacgatctgatctggaactactgggtcaacaattacctgctaggcaacgaaccgccggtgttcgacatcctgttctggaacaacgacaccacacggttgcccgcggcgttccacggcgacctgatcgaactgttcaaaaataacccactgattcgcccgaatgcactggaagtgtgcggcacccccatcgacctcaagcaggtgacggccgacatcttttccctggccggcaccaacgaccacatcaccccgtggaagtcctgctacaagtcggcgcaactgtttggcggcaacgttgaattcgtgctgtcgagcagcgggcatatccagagcatcctgaacccgccgggcaatccgaaatcgcgctacatgaccagcaccgaagtggcggaaaatgccgatgaatggcaagcgaatgccaccaagcatacagattcctggtggctgcactggcaggcctggcaggcccaacgctcgggcgagctgaaaaagtccccgacaaaactgggcagcaaggcgtatccggcaggtgaagcggcgccaggcacgtacgtgcacgaacgg

SEQ ID NO: 76 - protein, 559 aa, Pseudomonas sp.
MSNKSNDELKYQASENTLGLNPVVGLRGKDLLASARMVLRQAIKQPVHSVKHVAHFGLELKNVLLGKSGLQPTSDDRRFADPAWSQNPLYKRYLQTYLAWRKELHDWIDESNLAPKDVARGHFVINLMTEAMAPTNTAANPAAVKRFFETGGKSLLDGLSHLAKDLVHNGGMPSQVNMGAFEVGKSLGVTEGAVVFRNDVLELIQYKPTTEQVYERPLLVVPPQINKFYVFDLSPDKSLARFCLRNNVQTFIVSWRNPTKEQREWGLSTYIEALKEAVDVVTAITGSKDVNMLGACSGGITCTALLGHYAAIGENKVNALTLLVSVLDTTLDSDVALFVNEQTLEAAKRHSYQAGVLEGRDMAKVFAWMRPNDLIWNYWVNNYLLGNEPPVFDILFWNNDTTRLPAAFHGDLIELFKNNPLIRPNALEVCGTPIDLKQVTADIFSLAGTNDHITPWKSCYKSAQLFGGNVEFVLSSSGHIQSILNPPGNPKSRYMTSTEVAENADEWQANATKHTDSWWLHWQAWQAQRSGELKKSPTKLGSKAYPAGEAAPGTYVHER

SEQ ID NO: 77 - DNA, 1572 bp, Clostridium propionicum
atgagaaaggttcccattattaccgcagatgaggctgcaaagcttattaaagacggtgatacagttacaacaagtggtttcgttggaaatgcaatccctgaggctcttgatagagctgtagaaaaaagattcttagaaacaggcgaacccaaaaacattacctatgtttattgtggttctcaaggtaacagagacggaagaggtgctgagcactttgctcatgaaggccttttaaaacgttacatcgctggtcactgggctacagttcctgctttgggtaaaatggctatggaaaataaaatggaagcatataatgtatctcagggtgcattgtgtcatttgttccgtgatatagcttctcataagccaggcgtatttacaaaggtaggtatcggtactttcattgaccccagaaatggcggcggtaaagtaaatgatattaccaaagaagatattgttgaattggtagagattaagggtcaggaatatttattctaccctgcttttcctattcatgtagctcttattcgtggtacttacgctgatgaaagcggaaatatcacatttgagaaagaagttgctcctctggaaggaacttcagtatgccaggctgttaaaaacagtggcggtatcgttgtagttcaggttgaaagagtagtaaaagctggtactcttgaccctcgtcatgtaaaagttccaggaatttatgttgactatgttgttgttgctgacccagaagatcatcagcaatctttagattgtgaatatgatcctgcattatcaggcgagcatagaagacctgaagttgttggagaaccacttcctttgagtgcaaagaaagttattggtcgtcgtggtgccattgaattagaaaaagatgttgctgtaaatttaggtgttggtgcgcctgaatatgtagcaagtgttgctgatgaagaaggtatcgttgattttatgactttaactgctgaaagtggtgctattggtggtgttcctgctggtggcgttcgctttggtgcttcttataatgcggatgcattgatcgatcaaggttatcaattcgattactatgatggcggcggcttagacctttgctatttaggcttagctgaatgcgatgaaaaaggcaatatcaacgtttcaagatttggccctcgtatcgctggttgtggtggtttcatcaacattacacagaatacacctaaggtattcttctgtggtactttcacagcaggtggcttaaaggttaaaattgaagatggcaaggttattattgttcaagaaggcaagcagaaaaaattcttgaaagctgttgagcagattacattcaatggtgacgttgcacttgctaataagcaacaagtaacttatattacagaaagatgcgtattccttttgaaggaagatggtttgcacttatctgaaattgcacctggtattgatttgcagacacagattcttgacgttatggattttgcacctattattgacagagatgcaaacggccaaatcaaattgatggacgctgctttgtttgcagaaggcttaatgggtctgaaggaaatgaagtcc

SEQ ID NO: 78 - protein, 524 aa, Clostridium propionicum
MRKVPIITADEAAKLIKDGDTVTTSGFVGNAIPEALDRAVEKRFLETGEPKNITYVYCGSQGNRDGRGAEHFAHEGLLKRYIAGHWATVPALGKMAMENKMEAYNVSQGALCHLFRDIASHKPGVFTKVGIGTFIDPRNGGGKVNDITKEDIVELVEIKGQEYLFYPAFPIHVALIRGTYADESGNITFEKEVAPLEGTSVCQAVKNSGGIVVVQVERVVKAGTLDPRHVKVPGIYVDYVVVADPEDHQQSLDCEYDPALSGEHRRPEVVGEPLPLSAKKVIGRRGAIELEKDVAVNLGVGAPEYVASVADEEGIVDFMTLTAESGAIGGVPAGGVRFGASYNADALIDQGYQFDYYDGGGLDLCYLGLAECDEKGNINVSRFGPRIAGCGGFINITQNTPKVFFCGTFTAGGLKVKIEDGKVIIVQEGKQKKFLKAVEQITFNGDVALANKQQVTYITERCVFLLKEDGLHLSEIAPGIDLQTQILDVMDFAPIIDRDANGQIKLMDAALFAEGLMGLKEMKS

(19) United States
(12) Patent Application Publication
(10) Pub. No.: US 2012/0171737 A1
(43) Pub. Date: Jul. 5, 2012

(54) PREPARATION METHOD OF LACTATE POLYMERS AND LACTATE COPOLYMERS USING POLYHYDROXYALKANOATE SYNTHASE MUTANTS

(75) Inventors: Sang Yup Lee, Daejeon (KR); Yu Kyung Jung, Daejeon (KR); Taek Ho Yang, Daejeon Metropolitan City (KR); Hye Ok Kang, Daejeon Metropolitan City (KR)

(73) Assignees: KOREA ADVANCED INSTITUTE OF SCIENCE AND TECHNOLOGY, Daejeon (KR); LG CHEM, LTD, Seoul (KR)

(21) Appl. No.: 13/381,340

(22) PCT Filed: Jun. 30, 2010
(86) PCT No.: PCT/KR10/04240

§ 371 Date: Mar. 14, 2012

(30) Foreign Application Priority Data
Jun. 30, 2009 (KR) 10-2009-0059488

(51) Int. Cl. C12P 7/62; C07H 21/04; C12N 15/63; C12N 9/00; C12N 1/21

(52) U.S. Cl. 435/135; 435/183; 435/252.3; 435/252.31; 435/252.33; 435/252.34; 435/320.1; 536/23.2

(57) ABSTRACT

Mutants of various polyhydroxyalkanoate (PHA) synthases capable of synthesizing a lactate polymer (PLA) and a lactate copolymer (PLA copolymer), and a method of preparing a lactate polymer and a lactate copolymer using the same are provided. More specifically, a mutant of polyhydroxyalkanoate synthase set forth in SEQ ID NO: 2, 4, 6, or 8, and a method of preparing lactate polymer and lactate copolymer using the mutant of synthase are provided. The polyhydroxyalkanoate synthase set forth in SEQ ID NO: 2, 4, 6, or 8 can have an activity of synthesizing a lactate polymer and a lactate copolymer by an amino acid sequence mutation affecting an activity of synthesizing a lactate polymer, and can produce a lactate polymer and a copolymer that have different features, respectively, by using the mutants of the synthase.

### FIELD OF THE INVENTION

The present invention relates to mutants of various polyhydroxyalkanoate synthases capable of synthesizing a lactate polymer and a lactate copolymer using lactyl-CoA as a substrate. Also, the present invention relates to a method of preparing a lactate polymer and a lactate copolymer using mutants of polyhydroxyalkanoate synthases having an increased activity of synthesizing a lactate polymer and a lactate copolymer.

### DISCUSSION OF RELATED ART

Polylactate (PLA) is a typical biodegradable polymer derived from lactate and having high applicability as a polymer for medical use or a general-purpose polymer. PLA is now being prepared by polymerization of lactate produced from microorganism fermentation, but only PLA with low molecular weight (1000-5000 Dalton) is produced by direct polymerization of lactate. There is a method of polymerizing PLA with high molecular weight using a chain coupling agent from PLA with low molecular weight obtained from direct polymerization of lactate in order to synthesize PLA with at least 100,000 daltons. However, the above-mentioned method has disadvantages in that adding an organic solvent and the chain coupling agent makes the process more complex and they are also difficult to remove. As a process of producing PLA with high molecular weight that is now commercialized, there is a method of synthesizing PLA through a ring-opening condensation reaction of a lactide ring after converting lactate into lactide.

When PLA is synthesized from lactate through a chemical synthesis, PLA homopolymer can be easily obtained, but synthesis of PLA copolymer having various monomer compositions is difficult and has very low industrial efficiency.

Meanwhile, polyhydroxyalkanoate (PHA) is a polyester, which is accumulated inside a microorganism as energy or a reserve material of a carbon source when there is an excessive carbon source, but there is a lack of other nutrients, such as phosphorus, nitrogen, magnesium, oxygen, and the like. Since PHA has the similar physical properties as a synthetic polymer derived from conventional petroleum, and also has complete biodegradability, it is recognized as an alternative material of the conventional synthetic plastic.

An enzyme that converts metabolites of a microorganism into PHA monomer and PHA synthase, which synthesizes PHA polymer using PHA monomer, is required to produce PHA in a microorganism. Also, the same system is required to synthesize PLA and PLA copolymers using a microorganism, and an enzyme capable of further providing lactyl-CoA is also required in addition to the enzyme capable of providing hydroxyacyl-CoA, which is a substrate of original PHA synthase.

That is, this shows that the introduction of a monomer-supplying enzyme capable of smoothly supplying lactyl-CoA by expressing an activated type without inhibiting cell growth and PHA synthase capable of effectively recognizing lactyl-CoA as a substrate is very important in order to effectively produce PLA and PLA copolymer using a microorganism.

### SUMMARY OF THE INVENTION

Accordingly, the present inventors investigated polyhydroxyalkanoate synthases with high homology of an amino acid sequence with polyhydroxyalkanoate synthase derived from Pseudomonas sp. 6-19 that has been used for the conventional system. Then, the present inventors confirmed that a lactate polymer and a copolymer thereof can be produced with high efficiency by cloning polyhydroxyalkanoate synthase from four typical Pseudomonas strains among the synthases with high homology and then preparing mutants that change amino acid sequences affecting a synthesis activity of the lactate polymer and lactate copolymer. Therefore, the present invention was completed based on these facts.

An object of the present invention is to provide a recombinant microorganism capable of producing a lactate polymer or copolymer thereof using the mutants of various polyhydroxyalkanoate synthases and a method of preparing a lactate polymer or a copolymer thereof using the recombinant microorganism.

In order to achieve the object, the present invention provides a mutant of polyhydroxyalkanoate synthase that synthesizes a lactate polymer or a lactate copolymer using lactyl-CoA as a substrate, in which the mutant has an amino acid sequence comprising at least one mutation selected from the group consisting of

a) E130D and Q481K;

b) E130D, S325T, and Q481K;

c) E130D, S477F, and Q481K;

d) E130D, S325T, S477F, and Q481K; and

e) E130D, S325T, S477G, and Q481K;

in an amino acid sequence of polyhydroxyalkanoate synthase that is set forth in SEQ ID NO: 2, 4, 6, or 8.

In addition, the present invention provides a gene encoding the mutant of polyhydroxyalkanoate synthase, a recombinant vector for a synthesis of a lactate polymer or a lactate copolymer comprising the gene, a transformant transformed with the recombinant vector, and a method of preparing a lactate polymer or a lactate copolymer comprising culturing the transformant.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 shows a diagram of a process of constructing a recombinant expression vector comprising Type II polyhydroxyalkanoate synthases having high amino acid sequence homology with polyhydroxyalkanoate synthase derived from Pseudomonas sp. MBEL 6-19 of the present invention and propionyl-CoA transferase mutant (Pct540 Cp) derived from Clostridium propionicum, and mutants of polyhydroxyalkanoate synthases with increased activity of synthesizing a lactate polymer and a lactate copolymer from the recombinant expression vector; and

FIG. 2 shows multiple alignments of amino acid sequences of the polyhydroxyalkanoate synthases derived from Pseudomonas sp. MBEL 6-19 used for the present invention, a polyhydroxyalkanoate synthase derived from Pseudomonas chlororaphis, a polyhydroxyalkanoate synthase derived from Pseudomonas putida KT2440, a polyhydroxyalkanoate synthase derived from Pseudomonas resinovorans, and a polyhydroxyalkanoate synthase derived from Pseudomonas aeruginosa PAO1, in which amino acid residues (Cys296, Asp451, His479) that are presumed to be catalytic residues are marked with “*” and amino acid residues (Glu130, Ser325, Ser477, Gln481) affecting a change of substrate specificity to lactyl-CoA are marked respectively with the numbers in the amino acid sequence.

### DETAILED DESCRIPTION OF EXEMPLARY EMBODIMENTS

The present invention provides a recombinant microorganism having an ability to produce a lactate polymer and a copolymer thereof, by comprising a lactyl-CoA supply enzyme gene, and a polyhydroxyalkanoate synthase gene derived from Pseudononas chlororaphis (SEQ ID NO: 1), a polyhydroxyalkanoate synthase gene derived from Pseudomonas putida KT2440 (SEQ ID NO: 3), a polyhydroxyalkanoate synthase gene derived from Pseudomonas resinovorans (SEQ ID NO: 5), or a polyhydroxyalkanoate synthase gene derived from Pseudomonas aeruginosa PA01 (SEQ ID NO: 7) at the same time.

The present invention provides a mutant of a polyhydroxyalkanoate synthase that synthesizes a lactate polymer or a lactate copolymer using lactyl-CoA as a substrate, in which the mutant has an amino acid sequence comprising at least one selected from the group consisting of

a) E130D and Q481K;

b) E130D, S325T, and Q481K;

c) E130D, S477F, and Q481K;

d) E130D, S325T, S477F, and Q481K; or

e) E130D, S325T, S477G, and Q481K;

in amino acid sequences of polyhydroxyalkanoate synthase that are set forth in SEQ ID NO: 2, 4, 6, or 8.

The E130D mutation refers to a mutation in which the 130^{th} amino acid glutamate is substituted with aspartic acid; the S325T mutation refers to a mutation in which the 325^{th} amino acid serine is substituted with threonine; the S477F mutation refers to a mutation in which the 477^{th} amino acid serine is substituted with phenylalanine; The Q481K mutation refers to a mutation in which the 481^{st} amino acid glutamine is substituted with lysine; and the S477G mutation refers to a mutation in which the 477^{th} amino acid serine is substituted with glycine. An activity of producing a lactate polymer or a lactate copolymer by the mutant of polyhydroxyalkanoate synthase may be newly formed or increased due to the mutations in the amino acid sequence as mentioned above.

The amino acid sequences of SEQ ID NOS: 2, 4, 6, and 8 refer to an amino acid sequence of polyhydroxyalkanoate synthase derived from P. chlororaphis, an amino acid sequence of polyhydroxyalkanoate synthase derived from P. putida KT2440, an amino acid sequence of polyhydroxyalkanoate synthase derived from P. resinovorans, and an amino acid sequence of polyhydroxyalkanoate synthase derived from P. aeruginosa PA01, respectively. The present inventors confirmed that the lactate polymer and the lactate copolymer can be produced with high efficiency using lactyl-CoA as a substrate when using the mutants of PHA synthases derived from the above-described four Pseudomonas strains. Therefore, the present invention was completed based on these facts.

Also, the present invention provides genes that encode the mutants of the polyhydroxyalkanoate synthases.

Also, the present invention provides a recombinant vector for synthesis of a lactate polymer or a copolymer, comprising the genes.

Also, for the present invention, the recombinant vector further includes a propionyl-CoA transferase gene derived from Clostridium propionicum (pct_{Cp}) of SEQ ID NO: 77. The propionyl-CoA transferase is an enzyme that converts lactate and 3-hydroxybutyrate into lactyl-CoA and 3-hydroxybutyrate-CoA, respectively.

Preferably, the propionyl-CoA transferase gene may include a mutant gene of propionyl CoA-transferase comprising at least one of mutation selected from the group consisting of

a) A1200G mutation in a base sequence of SEQ ID NO: 77;

b) T78C, T669C, A1125G, and T1158C mutations in a base sequence of SEQ ID NO: 77;

c) Gly335Asp mutation in an amino acid sequence of SEQ ID NO: 78 and A1200G mutation in a base sequence of SEQ ID NO: 77;

d) Ala243Thr mutation in an amino acid sequence of SEQ ID NO: 78 and A1200G mutation in a base sequence of SEQ ID NO: 77;

e) Asp65Gly mutation in an amino acid sequence of SEQ ID NO: 78 and T669C, A1125G, and T1158C mutations in a base sequence of SEQ ID NO: 77;

f) Asp257Asn mutation in an amino acid sequence of SEQ ID NO: 78 and A1200G mutation in a base sequence of SEQ ID NO: 77;

g) Asp65Asn mutation in an amino acid sequence of SEQ ID NO: 78 and T699C, A1125G, and T1158C mutations in a base sequence of SEQ ID NO: 77;

h) Thr199Ile mutation in an amino acid sequence of SEQ ID NO: 78 and T699C, A1125G, and T1159C mutations in a base sequence of SEQ ID NO: 77; and

i) Val193Ala mutation in an amino acid sequence of SEQ ID NO: 78 and T78C, T699C, and T1158C mutations in a base sequence of SEQ ID NO: 77.

In the base sequence of SEQ ID NO: 77, the A1200G mutation refers to a mutation in which the 1200^{th} base adenine is substituted with guanine; the T78C mutation refers to a mutation in which the 78^{th} base thymine is substituted with cytosine; the T669C mutation refers to a mutation in which the 669th base thymine is substituted with cytosine; the A1125G mutation refers to a mutation in which the 1125^{th} base adenine is substituted with guanine; and the T1158C mutation refers to a mutation in which the 1158^{th} base thymine is substituted with cytosine. In the amino acid sequence of SEQ ID NO: 78, the Gly335Asp mutation refers to a mutation in which the 335^{th} amino acid glycine is substituted with aspartic acid; the Ala243Thr mutation refers to a mutation in which the 257^{th} amino acid alanine is substituted with threonine; the Asp65Asn mutation refers to a mutation in which the 65^{th} amino acid aspartic acid is substituted with asparagine; the Thr199Ile mutation refers to a mutation in which the 199^{th} amino acid threonine is substituted with isoleucine; and the Val193Ala mutation refers to a mutation in which the 193^{th} amino acid valine is substituted with alanine.

Preferably, the mutant gene of the propionyl CoA-transferase may be the mutant gene of the propionyl CoA-transferase (pct 540 Cp) including the Val193Ala mutation in an amino acid sequence of SEQ ID NO: 78 and the T78C, T699C, and T1158C mutation in a base sequence of SEQ ID NO: 77.

In addition, the present invention provides a transformant transformed with any one among the recombinant vectors, and a transformant obtained by a transformation of the transformed strains without the propionyl-CoA transferase using any one of the above-mentioned recombinant vectors is included in the scope of the present invention.

The recombinant vector according to the present invention may be transformed into a proper host cell using a general method. Bacteria, yeast, fungi, and the like may be used as a host cell, but the present invention is not limited thereto. The preferred host cell according to the present invention is a prokaryotic cell, and preferably, E. coli. Examples of a suitable prokaryotic cells include E. coli DH5a, E. coli JM101, E. coli K12 294, E. coli W3110, E. coli X1776, E. coli XL-1Blue (Stratagene), E. coli B, and the like. However, an E. coli strain such as FMB101, NM522, NM538, and NM539, and prokaryotic cell of other species and genera may be also used. In addition to the above-described E. coli, Agrobacterium genus strains, such as Agrobacterium A4, Bacilli, such as Bacillus subtilis, another enterbacter, such as Salmonella typhimurium or Serratia marcescens, and various Pseudomonas genus strains may be used as a host cell, but the present invention is not limited thereto.

A target plant for a transformation that can be used for the present invention may be tobacco, a tomato, a chili, a bean, rice, corn, and the like, but the present invention is not limited thereto. In addition, even though the plant used for a transformation is a plant of sexual propagation, it can be understood by a person of ordinary skill in the art that the plant can be repeatedly reproduced nonsexually by a tissue culture, and the like.

Also, the present invention provides a method of preparing a lactate polymer or a copolymer thereof, comprising culturing the transformant.

More preferably, the present invention provides a method of preparing a lactate polymer or a lactate copolymer, in which the culturing is performed under an environment of containing hydroxyalkanoate, and the prepared copolymer is hydroxyalkanoate-co-3-lactate that is the copolymer comprising a hydroxyalkanoate monomer unit and a lactate monomer unit.

In the present invention, the copolymer refers to a dipolymer having two types of monomers, a terpolymer having three types of monomers, a tetrapolymer having four types of monomers, and the like.

In the present invention, the hydroxyalkanoate may be at least one selected from the group consisting of 3-hydroxybutyrate, 3-hydroxyvalerate, 4-hydroxybutyrate, (D)-3-hydroxycarboxylic acids with a medium chain length of 6 to 14 carbon numbers, 3-hydroxypropionic acid, 3-hydroxyhexanic acid, 3-hydroxyheptanoic acid, 3-hydroxyoctanoic acid, 3-hydroxynonanoic acid, 3-hydroxydecanoic acid, 3-hydroxyundecanoic acid, 3-hydroxydodecanoic acid, 3-hydroxytetradecanoic acid, 3-hydroxyhexadecanoic acid, 4-hydroxyvaleric acid, 4-hydroxyhexanoic acid, 4-hydroxyheptanoic acid, 4-hydroxyoctanoic acid, 4-hydroxydecanoic acid, 5-hydroxyvaleric acid, 5-hydroxyhexanoic acid, 6-hydroxydodecanoic acid, 3-hydroxy-4-pentenoic acid, 3-hydroxy-4-trans-hexenoic acid, 3-hydroxy-4-cis-hexenoic acid, 3-hydroxy-5-hyxenoic acid, 3-hydroxy-6-trans-octenoic acid, 3-hydroxy-6-cis octenoic acid, 3-hydroxy-7-octenoic acid, 3-hydroxy-8-nonenoic acid, 3-hydroxy-9-decenoic acid, 3-hydroxy-5-cis-dodecenoic acid, 3-hydroxy-5-cis-dodecenoic acid, 3-hydroxy-5-cis-tetradecenoic acid, 3-hydroxy-7-cis-tetradecenoic acid, 3-hydroxy-5,8-cis-cis-tetradecenoic acid, 3-hydroxy-4-methylvaleric acid, 3-hydroxy-4-methylhexanoic acid, 3-hydroxy-5-methylhexanoic acid, 3-hydroxy-6-methylhexanoic acid, 3-hydroxy-4-methyloctanoic acid, 3-hydroxy-5-methyloctanoic acid, 3-hydroxy-6-methyloctanoic acid, 3-hydroxy-7-methyloctanoic acid, 3-hydroxy-6-methylnonanoic acid, 3-hydroxy-7-methylnonanoic acid, 3-hydroxy-8-methylnonanoic acid, 3-hydroxy-7-methyldecanoic acid, 3-hydroxy-9-methyldecanoic acid, 3-hydroxy-7-methyl-6-octenoic acid, malic acid, 3-hydroxysuccinic acid-methylester, 3-hydroxyadipinic acid-methylester, 3-hydroxysuberic acid-methylester, 3-hydroxyazelaic acid-methylester, 3-hydroxysebacic acid-methylester, 3-hydroxysuberic acid-ethylester, 3-hydroxysebacic acid-ethylester, 3-hydroxypimelic acid-propylester, 3-hydroxysebacic acid-benzylester, 3-hydroxy-8-acetoxyoctanoic acid, 3-hydroxy-9-acetoxynonanoic acid, phenoxy-3-hydroxybutyric acid, phenoxy-3-hydroxyvaleric acid, phenoxy-3-hydroxyheptanoic acid, phenoxy-3-hydroxyoctanoic acid, para-cyanophenoxy-3-hydroxybutyric acid, para-cyanophenoxy-3-hydroxyvaleric acid, para-cyanophenoxy-3-hydroxyhexanoic acid, para-nitrophenoxy-3-hydroxyhexanoic acid, 3-hydroxy-5-phenylvaleric acid, 3-hydroxy-5-cyclohexylbutyric acid, 3,12-dihydroxydodecanoic acid, 3,8-dihydroxy-5-cis-tetradecenoic acid, 3-hydroxy-4,5-epoxydecanoic acid, 3-hydroxy-6,7-epoxydodecanoic acid, 3-hydroxy-8,9-epoxy-5,6-cis-tetradecanoic acid, 7-cyano-3-hydroxyheptanoic acid, 7-cyano-3-hydroxyheptanoic acid, 3-hydroxy-7-fluoroheptanoic acid, 3-hydroxy-9-fluorononanoic acid, 3-hydroxy-6-chlorohexanoic acid, 3-hydroxy-8-chlorooctanoic acid, 3-hydroxy-6-bromohexanoic acid, 3-hydroxy-8-bromooctanoic acid, 3-hydroxy-11-bromoundecanoic acid, 3-hydroxy-2-butenoic acid, 6-hydroxy-3-dodecenoic acid, 3-hydroxy-2-methylbutyric acid, 3-hydroxy-2-methylvaleric acid and 3-hydroxy-2,6-dimethyl-5-heptenoic acid.

For the present invention, the term “vector” refers to a DNA construct comprising a DNA sequence to be operably linked to a suitable control sequence that can express DNA inside a host. A vector may be a plasmid, a phage particle, or simply a latent genomic insert. When a vector is transformed into a suitable host, it may be replicated or functioned regardless of a host genome, or in some cases, it may be integrated into a genome itself. A plasmid is the type that is most generally used as a vector, and thus plasmid and vector are sometimes used interchangeably in the present invention. However, the present invention also includes other types of a vector having the same function as the function that is known or is to be known in the art.

The expression “expression control sequence” refers to a DNA sequence that is essential for expression of a coding sequence that is operably linked in a specific host organism. The control sequence includes a promoter for performing a transcription, any operator sequence for controlling the transcription, a sequence for encoding a suitable mRNA ribosome binding domain, and a sequence for controlling terminations of the transcription and translation. For example, the control sequence suitable for a prokaryotic cell includes a promoter, any operator sequence, and a ribosome binding domain. In the eukaryotic cell, the control sequence includes a promoter, a polyadenylated signal, and an enhancer. A factor that has the biggest impact on the expression level of gene in a plasmid is a promoter. An SRα promoter, a promoter derived from cytomegalovirus, and the like are preferably used as a promoter for high expression.

Any one of very various expression control sequences may be used as a vector in order to express a DNA sequence of the present invention. Examples of the useful expression control sequence include, for example, an initial promoter and a late promoter of SV40 or adenovirus, a lac system, a trp system, a TAC or TRC system, T3 and T7 promoters, a major operator of phage lambda and a promoter region, a control region of fd coding protein, promoters to 3-phosphoglycerate kinase or other glycol degradation enzymes, the promoters of the phosphase, for example, Pho5, a promoter of a yeast alpha-hybrid, and other sequences for an inducement and a constitution that are known for controlling gene expression of a prokaryotic cell or a eukaryotic cell, or viruses thereof, and combinations thereof.

Nucleic acid is operably linked when it is arranged with a functional relationship with other nucleic acid sequences. It may be a gene and control sequence(s) that is linked in a process that enables the gene expression when a proper molecule (for example, transcriptional activation protein) is linked to the control sequence(s). For example, DNA for a pre-sequence or a secretion leader is operably linked to DNA for a polypeptide when expressing a pre-protein participating in secretion of a polypeptide; a promoter or an enhancer is operably linked to a coding sequence when affecting transcription of a sequence; a ribosome binding domain is operably linked to a coding sequence when affecting transcription of a sequence; or a ribosome binding domain is operably linked to a coding sequence when it is arranged to be easily translated. Generally, “operably linked” refers to a contact of a linked DNA sequence, or that the secretion leader is contacted and presented in the leading frame. However, the enhancer is not required to contact. When the domain is not presented, a synthetic oligonucleotide adaptor or linker according to a general method is used as mentioned above.

The term “expression vector” used for the present invention generally refers to a double-strained DNA fragment as a general recombinant carrier inserted with a xenogeneic DNA fragment. Here, xenogeneic DNA refers to heterogeneous DNA that is natively undiscovered DNA in a host cell. The expression vector is inside the host cell, can be replicated regardless of host chromosome DNA, and may produce several copies of a vector and (xenogeneic) DNA inserted in the same.

As is known in the art, the relevant gene is operably linked to the transcription and translation expression control sequences that function inside a selected expression host in order to increase the expression level of a transformed gene in the host cell. Preferably, the expression control sequence and relevant gene are included in one expression vector comprising the bacteria selection marker and replication origin together. When the expression host is a eukaryotic cell, the expression vector should further include a useful expression marker in a eukaryotic expression host.

In the present invention, the recombinant vector may be various vectors comprising a plasmid vector, a bacteriophage vector, a cosmid vector, and a yeast artificial chromosome (YAC) vector. The plasmid vector is preferably used for the object of the present invention. A typical plasmid vector that can be used for the object has a structure comprising (a) a replication origin that allows a replication to be effectively performed to include hundreds of plasmid vectors per host cell, (b) an antibiotic-resistance gene that allows a host cell transformed with a plasmid vector to be selected, and (c) a restriction site of restriction enzyme that can be inserted with a foreign DNA fragment. Even if there is no suitable restriction site of a restriction enzyme, a vector and foreign DNA may be easily ligated when using the linker and the synthetic oligonucleotide adaptor according to a general method.

In addition, the transformation of a prokaryotic cell may be easily achieved using a calcium chloride method as described in section 1.82 of Sambrook et al., supra. Optionally, electroporation (Neumann et al., EMBO J., 1: 841 (1982)) may also be used for the transformation of the cells.

A transfection of a plant for preparing the plant comprising the gene of the converting enzyme and the gene of the synthase of the present invention may be achieved by a general method using Agrobacterium, a virus vector, and the like. For example, the Agrobacterium genus microorganism is transformed with the recombinant vector comprising the gene according to the present invention, and then the transformed Agrobacterium genus microorganism is transfected into a tissue, and the like of the target plant to obtain a transfected plant. More specifically, the transfected plant may be prepared by the steps of (a) transfecting a target plant by preculturing an explant of the target plant, and then co-culturing it with the transformed Agrobacterium; (b) obtaining callus by culturing the transfected explant in a callus induction medium; and (c) developing a shoot by cutting the obtained callus and then culturing it in a shoot induction medium.

The term “explant” in the present invention refers to a fragment of tissue cut from a plant, and includes cotyledon or hypocotyl. The explant used for the method of the present invention may be cotyledon or hypocotyl, and the cotyledon obtained through germinating in an MS medium after disinfecting and washing a plant seed is more preferably used.

Hereinafter, the present invention will be described in further detail with reference to Examples. Examples are only for illustrating more specifically, and the range of the present invention is not limited to Examples.

Especially, the following Examples disclose only synthesis of poly(3-hydroxybutyrate-co-lactate) (P(3HB-co-LA)) by adding 3-hydroxybutyrate (3-HB) to a synthesis of lactate copolymer using a mutant of Type II PHA synthase, but it is obvious to a person of ordinary skill in the art that the copolymer of the polyhydroxyalkanoate and lactate can be produced by adding polyhydroxyalkanoate in addition to 3-HB.

### Example 1

### Gene Cloning and Investigation of PHA Synthase with High Homology with Amino Acid Sequence of PHA Synthase Derived from Pseudomonas sp. MBEL 6-19

In order to investigate polyhydroxyalkanoate synthase with high homology of an amino acid sequence with polyhydroxyalkanoate synthase derived from Pseudomonas sp. MBEL 6-19 (KCTC 11027BP) (PhaC1_{Ps6-19}) used for the present invention, Basic Local Alignment Search Tool (BLAST) analysis provided by the National Center for Biotechnology Information (NCBI) was used, and the synthases showing a relatively high amino acid sequence homology among the results are shown in Table 1. All of the enzymes were included in the group of Type II polyhydroxyalkanoate synthase that is medium-chain-length (MCL)-PHA synthase for polymerization of the substrate with a relatively long carbon chain.

TABLE 1
Polyhydroxyalkanoate synthase showing high homology of amino
acid sequence with PhaC1_{Ps6-19}

| | | Amino | |
|---|---|---|---|
| | Nucleotide | acid | Genbank |
| Organism | identity | identity | accession no. |
| Pseudomonas sp. MBEL 6-19 | 100 | 100 | FJ626663 |
| Pseudomonas sp. 3Y2 | 95 | 97 | AY754343 |
| Pseudomonas fluorescens PfO-1 | 88 | 93 | CP000094 |
| Pseudomonas sp. KBOS 03 | 87 | 91 | AY790327 |
| Pseudomonas chlororaphis | 85 | 90 | AB049413 |
| Pseudomonas corrugata CFBP5454 | 85 | 89 | AY910767 |
| Pseudomonas sp. 61-3 | 84 | 89 | AB014758 |
| Pseudomonas fluorescens Pf-5 | 85 | 89 | CP000076 |
| Comamonas testosteroni | 83 | 87 | AY790326 |
| Burkholderia caryophylli | 82 | 86 | AF394660 |
| Aeromonas hydrophila | 80 | 82 | AY786298 |
| Pseudomonas putida KT2440 | 80 | 81 | AE015451 |
| Pseudomonas pseudoalcaligenes | 80 | 79 | AF336848 |
| HBQ06 | | | |
| Pseudomonas resinovorans | 80 | 81 | AF129396 |
| Pseudomonas sp. HJ-2 | 80 | 78 | AY370934 |
| Pseudomonas stutzeri 1317 | 79 | 79 | AY278219 |
| Pseudomonas aeruginosa PAO1 | 78 | 77 | AE004091 |

The following four typical polyhydroxyalkanoate synthases were selected for an experiment in order to implement the present invention among the polyhydroxyalkanoate synthases [P. chlororaphis (KCTC 12349), P. putida KT2440 (ATCC 47054), P. resinovorans (KCTC 12498), and P. aeruginosa PAO1 (KCTC 1637)]. The whole DNA was extracted from each Pseudomonas strain in order to isolate the gene of polyhydroxyalkanoate synthase used for the present invention, and cloned with PCR by designing a primer as shown in Table 2 based on the synthase gene sequences deposited in NCBI Genbank.

TABLE 2
Primers for Cloning Polyhydroxyalkanoate Synthases

| | | | Regcognition Site Insert |
|---|---|---|---|
| Synthase | Microorganism | Synthase Cloning Primer | Primer |
| PhaC1_{Pch} | P. chlororaphis | SEQ ID NOS: 11 & 12 | SEQ ID NOS: 9 & 10, 13 & 26 |
| PhaC1_{Ppu} | P. putida KT2440 | SEQ ID NOS: 15 & 16 | SEQ ID NOS: 9 & 14, 17 & 26 |
| PhaC1_{Pre} | P. resinovorans | Sequence Nos. 19 & 20 | SEQ ID NOS: 9 & 18, 21 & 26 |
| PhaC1_{Pae} | P. aeruginosa PAO1 | SEQ ID NOS: 23 & 24 | SEQ ID NOS: 9 & 22, 25 & 26 |

SEQ ID NO: 9: 5′ - gca atg ccc gga gcc ggg cta gct ag - 3′
SEQ ID NO: 10: 5′ - gtc atc gtt att ctt gtt act cat gat ttg att gtc tct ctg - 3′
SEQ ID NO: 11: 5′ - Cag aga gac aat caa atc atg agt aac aag aat aac gat gac - 3′
SEQ ID NO: 12: 5′ - gca ctc atg caa gcg tta acg ttc atg gac ata agt acc - 3′
SEQ ID NO: 13: 5′ - ggt act tat gtc cat gaa cgt taa cgc ttg cat gag tgc - 3′
SEQ ID NO: 14: 5′ - ctc atc gtt gtt ctt gtt act cat gat ttg att gtc tct ctg - 3′
SEQ ID NO: 15: 5′ - cag aga gac aat caa atc atg agt aac aag aac aac gat gag - 3′
SEQ ID NO: 16: 5′ - gca ctc atg caa gcg tca acg ctc gtg aac gta ggt g - 3′
SEQ ID NO: 17: 5′ - cac cta cgt tca cga gcg ttg acg ctt gca tga gtg c - 3′
SEQ ID NO: 18: 5′ - gtc ttc att gtt ctt gtt gct cat gat ttg att gtc tct ctg - 3′
SEQ ID NO: 19: 5′ - cag aga gac aat caa atc atg agc aac aag aac aat gaa gac - 3′
SEQ ID NO: 20: 5′ - gca ctc atg caa gcg tca tcg ctc gtg cac ata ggt g - 3′
SEQ ID NO: 21: 5′ - cac cta tgt gca cga gcg atg acg ctt gca tga gtg c - 3′
SEQ ID NO: 22: 5′ - ctc gtt att gtt ctt ctg act cat gat ttg att gtc tct ctg - 3′
SEQ ID NO: 23: 5′ - cag aga gac aat caa atc atg agt cag aag aac aat aac gag - 3′
SEQ ID NO: 24: 5′ - gca ctc atg caa gcg tca tcg ttc atg cac gta ggt tc - 3′
SEQ ID NO: 25: 5′ - gaa cct acg tgc atg aac gat gac gct tgc atg agt gc - 3′
SEQ ID NO: 26: 5′ - gaa att gtt atc cgc ctg cag g - 3′

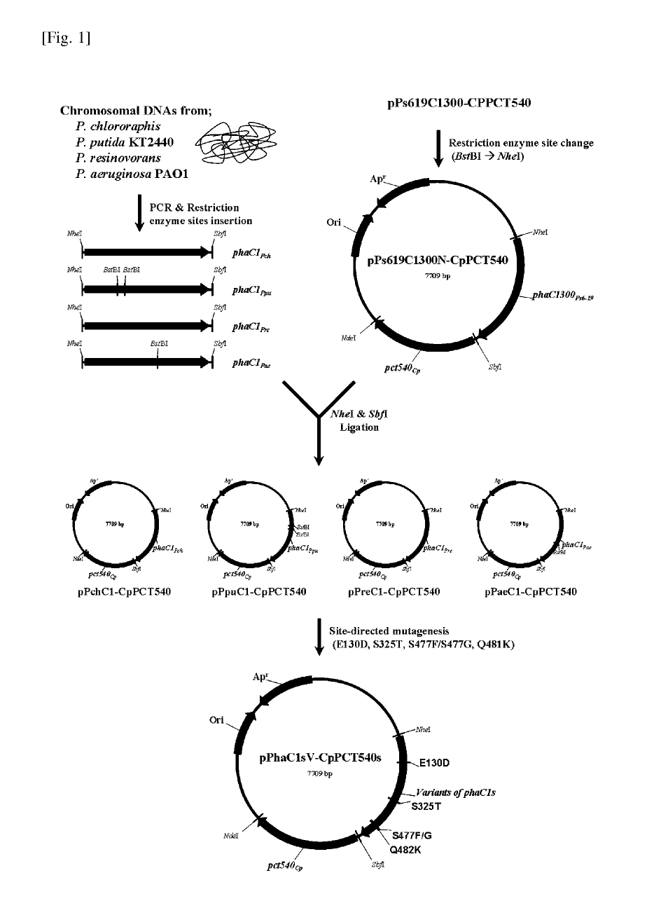

As a result of agarose gel electrophorosis of a PCR reactant, it was found that a gene fragment of 1.7 kbp size corresponding to the polyhydroxyalkanoate synthase gene was confirmed. For the expression of each synthase, a constitutive expression system was introduced, in which the system was in an operon type to express along with a mutant gene (pct540_{Cp}) of propionyl-CoA transferase derived from C. propionicum that is a monomer supply enzyme. Prior to this, it was found from the gene sequence of polyhydroxyalkanoate synthase deposited in NCBI Genbank that many BstBI sites used for a cloning site in the constitutive expression system in the conventional operon type were included in some polyhydroxyalkanoate synthase genes that were newly cloned. In order to solve the above problem, a pPs619C1300N-CpPCT540 vector in which a BstBI site was converted into an NheI site that was a unique cloning site was prepared based on pPs619C1300-CpPCT540 (WO09/022,797) using a site-directed mutagenesis (SDM) method (see FIG. 1.).

The prepared pPs619C1300N-CpPCT540 vector was cleaved with NheI/SbfI to remove phaC1300_{Ps6-19}, which is the conventional gene of polyhydroxyalkanoate synthase, and then the four obtained types of synthase genes were inserted to an NheI/SbfI recognition domain using SEQ ID NOS: 11 to 32 to complete each recombinant vector. The four prepared synthase gene PCR reactants were used as a template and an overlapping PCR was performed using “recognition site insert primer” as listed in Table 2 in order to prepare a polyhydroxyalkanoate synthase gene fragment including an RBS region upstream from a initiation codon while one NheI/SbfI recognition site was included one each end.

The base sequence of the synthase gene of the recombinant vector (pPchC1-CpPCT540, pPpuC1-CpPCT540, pPreC1-CpPCT540, pPaeC1-CpPCT540) comprising the four prepared polyhydroxyalkanoate synthase genes was confirmed by DNA sequencing (SEQ ID NOS: 1, 3, 5, and 7), and the amino acid sequences encoded by the same corresponded to SEQ ID NOS: 2, 4, 6, and 8, respectively. In the present invention, the gene sequences of the three synthases other than the polyhydroxyalkanoate synthase (PhaC1_{Pch}) derived from P. chlororaphis among the four newly cloned polyhydroxyalkanoate synthases were equal to the conventional genes deposited in NCBI Genbank (see Table 1), and PhaC1_{Pch} had several different nucleotide sequences that were thus deposited at Genbank (Accession no. FJ693714).

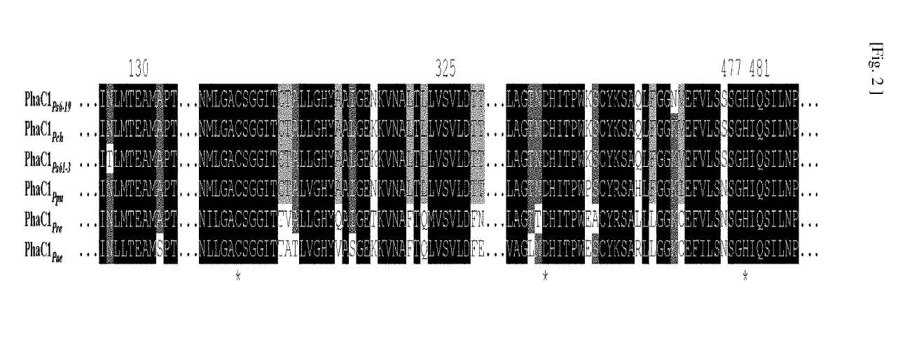

As a result of performing multiple alignment analysis of an amino acid sequence of polyhydroxyalkanoate synthase PhaC1Ps6-19 derived from Pseudomonas sp. 6-19 and amino acid sequences of the four polyhydroxyalkanoate synthases, it was confirmed that all amino acid residues (Glu130, Ser325, Ser477, Gln481) that are reported to affect a change in substrate specificity (WO08/062,999) to lactyl-CoA and the amino acid residues (Cys296, Asp451, His479) that are presumed to be catalytic residues were preserved in common (see FIG. 2). Also, it was confirmed that the polyhydroxyalkanoate synthase PhaC1Ps61-3 (Matsusaki et al., J. Bacteriol., 1998, 180:6459-6469; Gene sequence homology 84.3%, Amino acid sequence homology 88.7%) derived from Pseudomonas sp. strains 61-3 that is not described in Example of the present invention but shows high homology with PhaC1_{Ps6-19} also has an increased substrate specificity to lactyl-CoA and the above-mentioned catalytic residues, and high preservation of amino acid residues (see FIG. 2).

### Example 2

### Preparation of Mutant with High Activity of Production of Lactate Polymer and Copolymer

Type II polyhydroxyalkanoate synthase is known as an MCL-PHA synthase that polymerizes the substrate with a relatively long carbon number, and the studies on mutants with an increased activity of synthesis of short-chain-length (SCL)-PHA through researching various mutations were reported (WO08/062,999; Takase et al., J. Biochem., 2003, 133:139-145; Takase et al., Biomacromolecules, 2004, 5:480-485; Matsumoto et al., 2005, Biomacromolecules, 6:99-104; Matsumoto et al., 2006, Biomacromolecules, 7:2436-2442).

For the present invention, mutants as shown in the following Table 3 were prepared by introducing mutants of amino acid sequences that affect an activity of synthesis of a lactate polymer and a copolymer as confirmed from the previous invention (WO08/062,999) into four newly obtained Type II polyhydroxyalkanoate synthases (PhaC1_{Pch}, PhaC1_{Ppu}, PhaC1_{Pre}, PhaC1_{Pae}) using an SDM method with the primers of SEQ ID NOS: 27 to 74.

TABLE 3
Mutants of Type II polyhydroxyalkanoate Synthase

| | | Amino acid | |
|---|---|---|---|
| PHA Synthase | Recombinant Plasmid | Substitution | Primer or Source |
| PhaC1_{Ps6-19} | pPs619C1202-CpPCT540 | E130D | WO08/062999 |
| | | Q481K | |
| | pPs619C1310-CpPCT540 | E130D | WO08/062999 |
| | | S477F | |
| | | Q481K | |
| | pPs619C1301-CpPCT540 | E130D | WO08/062999 |
| | | S325T | |
| | | Q481K | |
| | pPs619C1339-CpPCT540 | E130D | WO08/062999 |
| | | S325T | |
| | | S477F | |
| | | Q481K | |
| | pPs619C1337-CpPCT540 | E130D | SEQ ID NOS: 27 & 28 |
| | | S325T | SEQ ID NOS: 29 & 30 |
| | | S477G | SEQ ID NOS: 31 & 32 |
| | | Q481K | SEQ ID NOS: 33 & 34 |
| PhaC1_{Pch} | pPchC1202-CpPCT540 | E130D | SEQ ID NOS: 35 & 36 |
| | | Q481K | SEQ ID NOS: 37 & 38 |
| | pPchC1310-CpPCT540 | E130D | SEQ ID NOS: 35 & 36 |
| | | S477F | SEQ ID NOS: 39 & 40 |
| | | Q481K | SEQ ID NOS: 37 & 38 |
| | pPchC1301-CpPCT540 | E130D | SEQ ID NOS: 35 & 36 |
| | | S325T | SEQ ID NOS: 41 & 42 |
| | | Q481K | SEQ ID NOS: 37 & 38 |
| | pPchC1339-CpPCT540 | E130D | SEQ ID NOS: 35 & 36 |
| | | S325T | SEQ ID NOS: 41 & 42 |
| | | S477F | SEQ ID NOS: 39 & 40 |
| | | Q481K | SEQ ID NOS: 37 & 38 |
| | pPchC1337-CpPCT540 | E130D | SEQ ID NOS: 35 & 36 |
| | | S325T | SEQ ID NOS: 41 & 42 |
| | | S477G | SEQ ID NOS: 43 & 44 |
| | | Q481K | SEQ ID NOS: 37 & 38 |
| PhaC1_{Ppu} | pPpuC1202-CpPCT540 | E130D | SEQ ID NOS: 45 & 46 |
| | | Q481K | SEQ ID NOS: 47 & 48 |
| | pPpuC1310-CpPCT540 | E130D | SEQ ID NOS: 45 & 46 |
| | | S477F | SEQ ID NOS: 49 & 50 |
| | | Q481K | SEQ ID NOS: 47 & 48 |
| | pPpuC1301-CpPCT540 | E130D | SEQ ID NOS: 45 & 46 |
| | | S325T | SEQ ID NOS: 51 & 52 |
| | | Q481K | SEQ ID NOS: 47 & 48 |
| | pPpuC1339-CpPCT540 | E130D | SEQ ID NOS: 45 & 46 |
| | | S325T | SEQ ID NOS: 51 & 52 |
| | | S477F | SEQ ID NOS: 49 & 50 |
| | | Q481K | SEQ ID NOS: 47 & 48 |
| | pPpuC1337-CpPCT540 | E130D | SEQ ID NOS: 45 & 46 |
| | | S325T | SEQ ID NOS: 51 & 52 |
| | | S477G | SEQ ID NOS: 53 & 54 |
| | | Q481K | SEQ ID NOS: 47 & 48 |
| PhaC1_{Pre} | pPreC1202-CpPCT540 | E130D | SEQ ID NOS: 55 & 56 |
| | | Q481K | SEQ ID NOS: 57 & 58 |
| | pPreC1310-CpPCT540 | E130D | SEQ ID NOS: 55 & 56 |
| | | S477F | SEQ ID NOS: 59 & 60 |
| | | Q481K | SEQ ID NOS: 57 & 58 |
| | pPreC1301-CpPCT540 | E130D | SEQ ID NOS: 55 & 56 |
| | | S325T | SEQ ID NOS: 61 & 62 |
| | | Q481K | SEQ ID NOS: 57 & 58 |
| | pPreC1339-CpPCT540 | E130D | SEQ ID NOS: 55 & 56 |
| | | S325T | SEQ ID NOS: 61 & 62 |
| | | S477F | SEQ ID NOS: 59 & 60 |
| | | Q481K | SEQ ID NOS: 57 & 58 |
| | pPreC1337-CpPCT540 | E130D | SEQ ID NOS: 55 & 56 |
| | | S325T | SEQ ID NOS: 61 & 62 |
| | | S477G | SEQ ID NOS: 63 & 64 |
| | | Q481K | SEQ ID NOS: 57 & 58 |
| PhaC1_{Pae} | pPaeC1202-CpPCT540 | E130D | SEQ ID NOS: 65 & 66 |
| | | Q481K | SEQ ID NOS: 67 & 68 |
| | pPaeC1310-CpPCT540 | E130D | SEQ ID NOS: 65 & 66 |
| | | S477F | SEQ ID NOS: 69 & 70 |
| | | Q481K | SEQ ID NOS: 67 & 68 |
| | pPaeC1301-CpPCT540 | E130D | SEQ ID NOS: 65 & 66 |
| | | S325T | SEQ ID NOS: 71 & 72 |
| | | Q481K | SEQ ID NOS: 67 & 68 |
| | pPaeC1339-CpPCT540 | E130D | SEQ ID NOS: 65 & 66 |
| | | S325T | SEQ ID NOS: 71 & 72 |
| | | S477F | SEQ ID NOS: 69 & 70 |
| | | Q481K | SEQ ID NOS: 67 & 68 |
| | pPaeC1337-CpPCT540 | E130D | SEQ ID NOS: 65 & 66 |
| | | S325T | SEQ ID NOS: 71 & 72 |
| | | S477G | SEQ ID NOS: 73 & 74 |
| | | Q481K | SEQ ID NOS: 67 & 68 |

SEQ ID NO: 27: 5′- atc aac ctc atg acc gat gcg atg gcg ccg acc - 3′
SEQ ID NO: 28: 5′- ggt cgg cgc cat cgc atc ggt cat gag gtt gat - 3′
SEQ ID NO: 29: 5′- ctg acc ttg ctg gtg acc gtg ctt gat acc acc - 3′
SEQ ID NO: 30: 5′- ggt ggt atc aag cac ggt cac cag caa ggt cag - 3′
SEQ ID NO: 31: 5′- gaa ttc gtg ctg tcg agc ggc ggg cat atc - 3′
SEQ ID NO: 32: 5′- gat atg ccc gcc gct cga cag cac gaa ttc - 3′
SEQ ID NO: 33: 5′- ggg cat atc aaa agc atc ctg aac ccg c - 3′
SEQ ID NO: 34: 5′- gcg ggt tca gga tgc ttt tga tat gcc c - 3′
SEQ ID NO: 35: 5′- atc aac ctg atg acc gat gcc atg gcg ccg acc - 3′
SEQ ID NO: 36: 5′- ggt cgg cgc cat ggc atc ggt cat cag gtt gat - 3′
SEQ ID NO: 37: 5′- ggg cat atc aaa agc atc ctc aac ccg c - 3′
SEQ ID NO: 38: 5′- gcg ggt tga gga tgc ttt tga tat gcc c - 3′
SEQ ID NO: 39: 5′- gaa ttc gtc ctc tcc agc ttt ggg cat atc - 3′
SEQ ID NO: 40: 5′- gat atg ccc aaa gct gga gag gac gaa ttc - 3′
SEQ ID NO: 41: 5′- ctg acc ctg ctg gtc acc gtg ctc gat acc acc - 3′
SEQ ID NO: 42: 5′- ggt ggt atc gag cac ggt gac cag cag ggt cag - 3′
SEQ ID NO: 43: 5′- gaa ttc gtc ctc tcc agc ggc ggg cat atc - 3′
SEQ ID NO: 44: 5′- gat atg ccc gcc gct gga gag gac gaa ttc - 3′
SEQ ID NO: 45: 5′- atc aac ctg atg acc gat gcc atg gcg ccg acc - 3′
SEQ ID NO: 46: 5′- ggt cgg cgc cat ggc atc ggt cat cag gtt gat - 3′
SEQ ID NO: 47: 5′- ggg cat atc aaa agc atc ctc aac ccg c - 3′
SEQ ID NO: 48: 5′- gcg ggt tga gga tgc ttt tga tat gcc c - 3′
SEQ ID NO: 49: 5′- gaa ttc gta ctg tcc aac ttt ggg cat atc - 3′
SEQ ID NO: 50: 5′- gat atg ccc aaa gtt gga cag tac gaa ttc - 3′
SEQ ID NO: 51: 5′- ctg acc ctg ctg gtc acc gtg ctg gac acc acc - 3′
SEQ ID NO: 52: 5′- ggt ggt gtc cag cac ggt gac cag cag ggt cag - 3′
SEQ ID NO: 53: 5′- gaa ttc gta ctg tcc aac ggc ggg cat atc - 3′
SEQ ID NO: 54: 5′- gat atg ccc gcc gtt gga cag tac gaa ttc - 3′
SEQ ID NO: 55: 5′- atc aac ctg atg acc gat gcg atg gcg ccc acc - 3′
SEQ ID NO: 56: 5′- ggt ggg cgc cat cgc atc ggt cat cag gtt gat - 3′
SEQ ID NO: 57: 5′- ggc cac atc aaa agc att ctc aac cca c - 3′
SEQ ID NO: 58: 5′- gtg ggt tga gaa tgc ttt tga tgt ggc c - 3′
SEQ ID NO: 59: 5′- gag ttc gtg ctg tcc aac ttt ggc cac atc - 3′
SEQ ID NO: 60: 5′- gat gtg gcc aaa gtt gga cag cac gaa ctc - 3′
SEQ ID NO: 61: 5′- ttc acc cag atg gtc acc gtg ctc gac ttc aac - 3′
SEQ ID NO: 62: 5′- gtt gaa gtc gag cac ggt gac cat ctg ggt gaa - 3′
SEQ ID NO: 63: 5′- gag ttc gtg ctg tcc aac ggc ggc cac atc - 3′
SEQ ID NO: 64: 5′- gat gtg gcc gcc gtt gga cag cac gaa ctc - 3′
SEQ ID NO: 65: 5′- atc aac ctg ctg acc gat gcg atg tcg ccg acc - 3′
SEQ ID NO: 66: 5′- ggt cgg cga cat cgc atc ggt cag cag gtt gat - 3′
SEQ ID NO: 67: 5′- ggt cac atc aaa agc atc ctc aac ccac - 3′
SEQ ID NO: 68: 5′- gtg ggt tga gga tgc ttt tga tgt gac c - 3′
SEQ ID NO: 69: 5′- gag ttc atc ctc tcc aac ttt ggt cac atc - 3′
SEQ ID NO: 70: 5′- gat gtg acc aaa gtt gga gag gat gaa ctc - 3′
SEQ ID NO: 71: 5′- ttc acc caa ctg gtc acc gtg ctc gac ttc gaa - 3′
SEQ ID NO: 72: 5′- ttc gaa gtc gag cac ggt gac cag ttg ggt gaa - 3′
SEQ ID NO: 73: 5′- gag ttc atc ctc tcc aac ggc ggt cac atc - 3′
SEQ ID NO: 74: 5′- gat gtg acc gcc gtt gga gag gat gaa ctc - 3′

The mutants of the synthase prepared from polyhydroxyalkanoate synthase derived from Pseudomonas sp. MBEL 6-19, PhaC1_{Ps6-19}, and four Type II polyhydroxyalkanoate synthases prepared according to the present invention were introduced into the constitutive expression system in an operon type that is expressed along with the mutant gene (pct540_{Cp}; WO09/022,797) of propionyl-CoA transferase, a monomer supply enzyme. In order to confirm that the prepared synthase mutants exhibit the activity of synthesis of a lactate copolymer [P(3HB-co-LA)], the recombinant vectors as listed in Table 3 were transformed to E. coli XL-1 Blue, and then grown in a P(3HB-co-LA) detection medium (LB agar, glucose 20 g/L, 3HB 2 g/L, Nile red 0.5 μg/mL). As a result, it was found that the lactate copolymer was produced in all of E. coli XL-1 Blue transformed with the recombinant vectors comprising the mutants of synthases with the amino acid mutations, even though it did differ slightly depending on the mutants, and the lactate copolymer was not produced in E. coli XL-1 Blue that expressed wild-type synthase without introducing the amino acid mutation. That is, as mentioned in Example 1, it was found that all of PhaC1_{Pch}, PhaC1_{Ppu}, PhaC1_{Pre}, and PhaC1Pae used for the present invention in addition to PhaC1_{Ps6-19} had newly formed or increased activities of production of a lactate copolymer due to the amino acid mutation in the positions of Glu130, Ser325, Ser477, and Gln481.

### Example 3

### Production of Lactate Polymer and Copolymer Using Synthase Mutant

In order to quantitatively analyze the activities of synthesis of five wild-type Type II polyhydroxyalkanoate synthases (PhaC1_{Ps6-19}, PhaC1_{Pch}, PhaC1_{Ppu}, PhaC1_{Pre}, PhaC1_{Pae}) prepared in Example 2 and lactate copolymer of mutants thereof [P(3HB-co-LA)], E. coli XL-1 Blue transformed with the recombinant expression vectors (Table 3) comprising the above-mentioned synthases was cultured in a flask containing MR medium supplemented with glucose (20 g/L) and 3HB (2 g/L) at 30° C. for 4 days. Compositions of MR medium used for the present invention were as listed in Table 4. The cultured bacteria was collected through centrifugation, washed three times with distilled water, and then dried in an oven at 100° C. for 24 hours. The compositions and content of the polymer synthesized in the dried cell were analyzed through gas chromatography, and the results are shown in Table 5.

TABLE 4
Composition of MR medium used for Culture of Recombinant E. coli

| | Modified | |
|---|---|---|
| Components | R (MR) (/L) | |
| KH₂PO₄ | 6.67 | g |
| (NH₄)₂HPO₄ | 4 | g |
| Citrate | 0.8 | g |
| MgSO₄•H₂O | 0.8 | g |
| Micro ingredient* | 5 | mL |

*Micro Ingredient (/L): FeSO₄•H₂O, 10 g; ZnSO₄•H₂O, 2.25 g; CuSO₄•H₂O, 1 g; MnSO₄•H₂O, 0.5 g; CaCl₂•H₂O, 2 g; Na₂B₄O₇•H₂O, 0.23 g; (NH₄)₆Mo₇O₂₄, 0.1 g; 35% HCl, 10 mL.

TABLE 5
Synthesis of Lactate Copolymer Using Synthase Mutant

| | | | Content of | |
|---|---|---|---|---|
| | | | Polymer | LA |
| Source | PHA Synthase | Amino acid substitution | (wt %) | Fragment |
| Pseudomonas sp. | PhaC1_{Ps6-19} | — | 0.6 ± 0.1 | 0 |
| MBEL 6-19 | PhaC1202_{Ps6-19} | E130D, Q481K | 39.8 ± 5.1 | 38.9 ± 1.7 |
| | PhaC1301_{Ps6-19} | E130D, S477F, Q481K | 49.9 ± 8.2 | 36.2 ± 3.3 |
| | PhaC1310_{Ps6-19} | E130D, S325T, Q481K | 50.9 ± 2.7 | 43.9 ± 1.8 |
| | PhaC1339_{Ps6-19} | E130D, S325T, S477F, Q481K | 59.4 ± 1.6 | 45.4 ± 3.6 |
| | PhaC1337_{Ps6-19} | E130D, S325T, S477G, Q481K | 55.4 ± 3.5 | 51.7 ± 2.6 |
| Pseudomonas | PhaC1_{Pch} | — | 0.2 ± 0 | 0 |
| chlororaphis | PhaC1202_{Pch} | E130D, Q481K | 13.2 ± 3.4 | 20.1 ± 0.4 |
| | PhaC1301_{Pch} | E130D, S477F, Q481K | 21.3 ± 3.5 | 18.8 ± 1.2 |
| | PhaC1310_{Pch} | E130D, S325T, Q481K | 27.5 ± 4.4 | 23.2 ± 7.6 |
| | PhaC1339_{Pch} | E130D, S325T, S477F, Q481K | 46.8 ± 3.1 | 34.6 ± 2.0 |
| | PhaC1337_{Pch} | E130D, S325T, S477G, Q481K | 52.8 ± 1.4 | 43.7 ± 1.8 |
| Pseudomonas | PhaC1_{Ppu} | — | 0 | 0 |
| putida KT2440 | PhaC1202_{Ppu} | E130D, Q481K | 6.8 ± 0.9 | 0 |
| | PhaC1301_{Ppu} | E130D, S477F, Q481K | 7.8 ± 0.5 | 7.1 ± 0.8 |
| | PhaC1310_{Ppu} | E130D, S325T, Q481K | 39.3 ± 0.6 | 27.5 ± 0.8 |
| | PhaC1339_{Ppu} | E130D, S325T, S477F, Q481K | 41.3 ± 3.1 | 25.1 ± 0.3 |
| | PhaC1337_{Ppu} | E130D, S325T, S477G, Q481K | 43.0 ± 2.2 | 32.0 ± 2.6 |
| Pseudomonas | PhaC1_{Pre} | — | 1.4 ± 0 | 39.8 ± 4.5 |
| resinovorans | PhaC1202_{Pre} | E130D, Q481K | 31.6 ± 4.4 | 62.7 ± 2.1 |
| | PhaC1301_{Pre} | E130D, S477F, Q481K | 30.8 ± 2.9 | 55.3 ± 2.3 |
| | PhaC1310_{Pre} | E130D, S325T, Q481K | 57.5 ± 0.4 | 49.0 ± 2.4 |
| | PhaC1339_{Pre} | E130D, S325T, S477F, Q481K | 52.1 ± 0.4 | 56.5 ± 0.3 |
| | PhaC1337_{Pre} | E130D, S325T, S477G, Q481K | 53.6 ± 2.9 | 65.5 ± 1.0 |
| Pseudomonas | PhaC1_{Pae} | — | 0 | 0 |
| aeruginosa PA01 | PhaC1202_{Pae} | E130D, Q481K | 5.1 ± 1.1 | 30.7 ± 3.1 |
| | PhaC1301_{Pae} | E130D, S477F, Q481K | 8.4 ± 0.6 | 21.5 ± 0.5 |
| | PhaC1310_{Pae} | E130D, S325T, Q481K | 36.0 ± 1.3 | 36.2 ± 0.1 |
| | PhaC1339_{Pae} | E130D, S325T, S477F, Q481K | 39.9 ± 1.1 | 40.1 ± 0.6 |
| | PhaC1337_{Pae} | E130D, S325T, S477G, Q481K | 49.2 ± 2.9 | 52.7 ± 0.5 |

As a result of gas chromatography analysis, while lactate copolymer was not produced or produced in a very small amount (1.4 wt %) in the recombinant E. coli XL-1 Blue that expressed five wild-type Type II polyhydroxyalkanoate synthases (PhaC1_{Ps6-19}, PhaC1_{Pch}, PhaC1_{Ppu}, PhaC1_{Pre}, PhaC1_{Pae}), a lactate copolymer [P(3HB-co-LA)] was accumulated in a cell because of the activity that can accept lactyl-CoA as a substrate was newly generated (or increased) in the case of the mutants with the amino acid mutations in the positions of Glu130, Ser325, Ser477, and Gln481. For the compositions and content of the synthesized lactate copolymer in a cell, the mutants introduced with the mutations of E130D, S325T, S477G, and Q481K at the same time exhibited a high accumulation rate of a polymer in a cell and high synthesis activity of a copolymer, thereby showing high content of lactate, depending on a type of each polyhydroxyalkanoate synthase. Also, the synthesis activity of a lactate polymer of the mutants introduced with E130D and Q481K and the mutants introduced with E130D, S325T, S477G, and Q481K at the same time was analyzed among the mutants as listed in Table 3. To achieve this, E. coli XL-1 Blue that expressed each mutation was cultured in a flask comprising MR medium supplemented with glucose (20 g/L) at 30° C. for 4 days. As an analysis result, the mutants with all of E130D, S325T, S477G, and Q481K produced about 2 to 7 wt % lactate polymer when it was used to synthesize the lactate polymer, and the activity of the mutant of synthase derived from P. resinovorans was the highest (see Table 6).

TABLE 6
Synthesis of Lactate Polymer using Synthase Mutant

| Source | PHA Synthase | Amino acid substitution | PLA (wt %) |
|---|---|---|---|
| Pseudomonas | PhaC1202_{Ps6-19} | E130D, Q481K | 0.5 ± 0.1 |
| sp. MBEL 6-19 | | | |
| | PhaC1337_{Ps6-19} | E130D, S325T, S477G, | 7.2 ± 0.3 |
| | | Q481K | |
| Pseudomonas | PhaC1202_{Pch} | E130D, Q481K | 00.3± |
| chlororaphis | | | |
| | PhaC1337_{Pch} | E130D, S325T, S477G, | 4.5 ± 0.4 |
| | | Q481K | |
| Pseudomonas | PhaC1202_{Ppu} | E130D, Q481K | 0 |
| putida KT2440 | | | |
| | PhaC1337_{Ppu} | E130D, S325T, S477G, | 2.0 ± 0.1 |
| | | Q481K | |
| Pseudomonas | PhaC1202_{Pre} | E130D, Q481K | 1.2 ± 0.3 |
| resinovorans | | | |
| | PhaC1337_{Pre} | E130D, S325T, S477G, | 7.3 ± 0 |
| | | Q481K | |
| Pseudomonas | PhaC1202_{Pae} | E130D, Q481K | 0.6 ± 0.3 |
| aeruginosa | | | |
| PAO1 | | | |
| | PhaC1337_{Pae} | E130D, S325T, S477G, | 5.9 ± 0.4 |
| | | Q481K | |

All of the polyhydroxyalkanoate synthases according to the present invention can have an activity of synthesizing a lactate polymer and a lactate copolymer by an amino acid sequence mutation affecting the activity of synthesizing a lactate polymer and a lactate copolymer, and then can produce a lactate copolymer having different features, respectively, when using the mutants of synthases.

While the invention has been shown and described with reference to certain exemplary embodiments thereof, it will be understood by those skilled in the art that various changes in form and details may be made therein without departing from the spirit and scope of the invention as defined by the appended claims.

1. A mutant of polyhydroxyalkanoate synthase for synthesizing a lactate polymer or a lactate copolymer using lactyl-CoA as a substrate, in which the mutant has at least one of mutation selected from the group consisting of
a) E130D and Q481K;
b) E130D, S325T, and Q481K;
c) E130D, S477F, and Q481K;
d) E130D, S325T, S477F, and Q481K; and
e) E130D, S325T, S477G and Q481K;
in an amino acid sequence of polyhydroxyalkanoate synthase set forth in SEQ ID NO: 2, 4, 6 or 8.

2. A gene encoding the mutant of polyhydroxyalkanoate synthase of claim 1.

3. A recombinant vector for synthesizing a lactate polymer or a lactate copolymer comprising the gene of claim 2.

4. The recombinant vector of claim 3, further comprising a propionyl-CoA transferase gene (pct) of SEQ ID NO: 77.

5. The recombinant vector of claim 4, wherein the propionyl-CoA transferase gene of SEQ ID NO: 77 includes the propionyl CoA-transferase mutant gene comprising the mutation selected from the group consisting of
a) A1200G mutation in a base sequence of SEQ ID NO: 77;
b) T78C, T669C, A1125G, and T1158C mutations in a base sequence of SEQ ID NO: 77;
c) Gly335Asp mutation in an amino acid sequence of SEQ ID NO: 78 and A1200G mutations in a base sequence of SEQ ID NO: 77;
d) Ala243Thr mutation in an amino acid sequence of SEQ ID NO: 78 and A1200G mutation in a base sequence of SEQ ID NO: 77;
e) Asp65Gly mutation in an amino acid sequence of SEQ ID NO: 78 and T669C, A1125G, and T1158C mutations in a base sequence of SEQ ID NO: 77;
f) Asp257Asn mutation in an amino acid sequence of SEQ ID NO: 78 and A1200G mutation in a base sequence of SEQ ID NO: 77;
g) Asp65Asn mutation in an amino acid sequence of SEQ ID NO: 78 and T699C, A1125G, and T1158C mutations in a base sequence of SEQ ID NO: 77;
h) Thr199Ile mutation in an amino acid sequence of SEQ ID NO: 78 and T699C, A1125G, and T1159C mutations in a base sequence of SEQ ID NO: 77; and
i) Val193Ala mutation in an amino acid sequence of SEQ ID NO: 78 and T78C, T699C, and T1158C mutations in a base sequence of SEQ ID NO: 77.

6. A transformant transformed with the recombinant vector of claim 3.

7. A method of preparing a lactate polymer or a lactate copolymer, comprising culturing the transformant of claim 6.

8. The method of claim 7, wherein the culturing is performed in an environment containing hydroxyalkanoate and the copolymer is hydroxyalkanoate-co-3-lactate.

9. The method of claim 8, wherein the hydroxyalkanoate is at least one selected from the group consisting of 3-hydroxybutyrate, 3-hydroxyvalerate, 4-hydroxybutyrate, (D)-3-hydroxycarboxylic acids with a medium chain length of 6 to 14 carbon numbers, 3-hydroxypropionic acid, 3-hydroxyhexanic acid, 3-hydroxyheptanoic acid, 3-hydroxyoctanoic acid, 3-hydroxynonanoic acid, 3-hydroxydecanoic acid, 3-hydroxyundecanoic acid, 3-hydroxydodecanoic acid, 3-hydroxytetradecanoic acid, 3-hydroxyhexadecanoic acid, 4-hydroxyvaleric acid, 4-hydroxyhexanoic acid, 4-hydroxyheptanoic acid, 4-hydroxyoctanoic acid, 4-hydroxydecanoic acid, 5-hydroxyvaleric acid, 5-hydroxyhexanoic acid, 6-hydroxydodecanoic acid, 3-hydroxy-4-pentenoic acid, 3-hydroxy-4-trans-hexenoic acid, 3-hydroxy-4-cis-hexenoic acid, 3-hydroxy-5-hyxenoic acid, 3-hydroxy-6-trans-octenoic acid, 3-hydroxy-6-cis octenoic acid, 3-hydroxy-7-octenoic acid, 3-hydroxy-8-nonenoic acid, 3-hydroxy-9-decenoic acid, 3-hydroxy-5-cis-dodecenoic acid, 3-hydroxy-5-cis-dodecenoic acid, 3-hydroxy-5-cis-tetradecenoic acid, 3-hydroxy-7-cis-tetradecenoic acid, 3-hydroxy-5,8-cis-cis-tetradecenoic acid, 3-hydroxy-4-methylvaleric acid, 3-hydroxy-4-methylhexanoic acid, 3-hydroxy-5-methylhexanoic acid, 3-hydroxy-6-methylhexanoic acid, 3-hydroxy-4-methyloctanoic acid, 3-hydroxy-5-methyloctanoic acid, 3-hydroxy-6-methyloctanoic acid, 3-hydroxy-7-methyloctanoic acid, 3-hydroxy-6-methylnonanoic acid, 3-hydroxy-7-methylnonanoic acid, 3-hydroxy-8-methylnonanoic acid, 3-hydroxy-7-methyldecanoic acid, 3-hydroxy-9-methyldecanoic acid, 3-hydroxy-7-methyl-6-octenoic acid, malic acid, 3-hydroxysuccinic acid-methylester, 3-hydroxyadipinic acid-methylester, 3-hydroxysuberic acid-methylester, 3-hydroxyazelaic acid-methylester, 3-hydroxysebacic acid-methylester, 3-hydroxysuberic acid-ethylester, 3-hydroxysebacic acid-ethylester, 3-hydroxypimelic acid-propylester, 3-hydroxysebacic acid-benzylester, 3-hydroxy-8-acetoxyoctanoic acid, 3-hydroxy-9-acetoxynonanoic acid, phenoxy-3-hydroxybutyric acid, phenoxy-3-hydroxyvaleric acid, phenoxy-3-hydroxyheptanoic acid, phenoxy-3-hydroxyoctanoic acid, para-cyanophenoxy-3-hydroxybutyric acid, para-cyanophenoxy-3-hydroxyvaleric acid, para-cyanophenoxy-3-hydroxyhexanoic acid, para-nitrophenoxy-3-hydroxyhexanoic acid, 3-hydroxy-5-phenylvaleric acid, 3-hydroxy-5-cyclohexylbutyric acid, 3,12-dihydroxydodecanoic acid, 3,8-dihydroxy-5-cis-tetradecenoic acid, 3-hydroxy-4,5-epoxydecanoic acid, 3-hydroxy-6,7-epoxydodecanoic acid, 3-hydroxy-8,9-epoxy-5,6-cis-tetradecanoic acid, 7-cyano-3-hydroxyheptanoic acid, 7-cyano-3-hydroxyheptanoic acid, 3-hydroxy-7-fluoroheptanoic acid, 3-hydroxy-9-fluorononanoic acid, 3-hydroxy-6-chlorohexanoic acid, 3-hydroxy-8-chlorooctanoic acid, 3-hydroxy-6-bromohexanoic acid, 3-hydroxy-8-bromooctanoic acid, 3-hydroxy-11-bromoundecanoic acid, 3-hydroxy-2-butenoic acid, 6-hydroxy-3-dodecenoic acid, 3-hydroxy-2-methylbutyric acid, 3-hydroxy-2-methylvaleric acid and 3-hydroxy-2,6-dimethyl-5-heptenoic acid.

10. A transformant transformed with the recombinant vector of claim 4.

11. A transformant transformed with the recombinant vector of claim 5.

12. A method of preparing a lactate polymer or a lactate copolymer, comprising culturing the transformant of claim 10.

13. A method of preparing a lactate polymer or a lactate copolymer, comprising culturing the transformant of claim 11.

14. The method of claim 12, wherein the culturing is performed in an environment containing hydroxyalkanoate and the copolymer is hydroxyalkanoate-co-3-lactate.

15. The method of claim 13, wherein the culturing is performed in an environment containing hydroxyalkanoate and the copolymer is hydroxyalkanoate-co-3-lactate.

16. The method of claim 14, wherein the hydroxyalkanoate is at least one selected from the group consisting of 3-hydroxybutyrate, 3-hydroxyvalerate, 4-hydroxybutyrate, (D)-3-hydroxycarboxylic acids with a medium chain length of 6 to 14 carbon numbers, 3-hydroxypropionic acid, 3-hydroxyhexanic acid, 3-hydroxyheptanoic acid, 3-hydroxyoctanoic acid, 3-hydroxynonanoic acid, 3-hydroxydecanoic acid, 3-hydroxyundecanoic acid, 3-hydroxydodecanoic acid, 3-hydroxytetradecanoic acid, 3-hydroxyhexadecanoic acid, 4-hydroxyvaleric acid, 4-hydroxyhexanoic acid, 4-hydroxyheptanoic acid, 4-hydroxyoctanoic acid, 4-hydroxydecanoic acid, 5-hydroxyvaleric acid, 5-hydroxyhexanoic acid, 6-hydroxydodecanoic acid, 3-hydroxy-4-pentenoic acid, 3-hydroxy-4-trans-hexenoic acid, 3-hydroxy-4-cis-hexenoic acid, 3-hydroxy-5-hyxenoic acid, 3-hydroxy-6-trans-octenoic acid, 3-hydroxy-6-cis octenoic acid, 3-hydroxy-7-octenoic acid, 3-hydroxy-8-nonenoic acid, 3-hydroxy-9-decenoic acid, 3-hydroxy-5-cis-dodecenoic acid, 3-hydroxy-5-cis-dodecenoic acid, 3-hydroxy-5-cis-tetradecenoic acid, 3-hydroxy-7-cis-tetradecenoic acid, 3-hydroxy-5,8-cis-cis-tetradecenoic acid, 3-hydroxy-4-methylvaleric acid, 3-hydroxy-4-methylhexanoic acid, 3-hydroxy-5-methylhexanoic acid, 3-hydroxy-6-methylhexanoic acid, 3-hydroxy-4-methyloctanoic acid, 3-hydroxy-5-methyloctanoic acid, 3-hydroxy-6-methyloctanoic acid, 3-hydroxy-7-methyloctanoic acid, 3-hydroxy-6-methylnonanoic acid, 3-hydroxy-7-methylnonanoic acid, 3-hydroxy-8-methylnonanoic acid, 3-hydroxy-7-methyldecanoic acid, 3-hydroxy-9-methyldecanoic acid, 3-hydroxy-7-methyl-6-octenoic acid, malic acid, 3-hydroxysuccinic acid-methylester, 3-hydroxyadipinic acid-methylester, 3-hydroxysuberic acid-methylester, 3-hydroxyazelaic acid-methylester, 3-hydroxysebacic acid-methylester, 3-hydroxysuberic acid-ethylester, 3-hydroxysebacic acid-ethylester, 3-hydroxypimelic acid-propylester, 3-hydroxysebacic acid-benzylester, 3-hydroxy-8-acetoxyoctanoic acid, 3-hydroxy-9-acetoxynonanoic acid, phenoxy-3-hydroxybutyric acid, phenoxy-3-hydroxyvaleric acid, phenoxy-3-hydroxyheptanoic acid, phenoxy-3-hydroxyoctanoic acid, para-cyanophenoxy-3-hydroxybutyric acid, para-cyanophenoxy-3-hydroxyvaleric acid, para-cyanophenoxy-3-hydroxyhexanoic acid, para-nitrophenoxy-3-hydroxyhexanoic acid, 3-hydroxy-5-phenylvaleric acid, 3-hydroxy-5-cyclohexylbutyric acid, 3,12-dihydroxydodecanoic acid, 3,8-dihydroxy-5-cis-tetradecenoic acid, 3-hydroxy-4,5-epoxydecanoic acid, 3-hydroxy-6,7-epoxydodecanoic acid, 3-hydroxy-8,9-epoxy-5,6-cis-tetradecanoic acid, 7-cyano-3-hydroxyheptanoic acid, 7-cyano-3-hydroxyheptanoic acid, 3-hydroxy-7-fluoroheptanoic acid, 3-hydroxy-9-fluorononanoic acid, 3-hydroxy-6-chlorohexanoic acid, 3-hydroxy-8-chlorooctanoic acid, 3-hydroxy-6-bromohexanoic acid, 3-hydroxy-8-bromooctanoic acid, 3-hydroxy-11-bromoundecanoic acid, 3-hydroxy-2-butenoic acid, 6-hydroxy-3-dodecenoic acid, 3-hydroxy-2-methylbutyric acid, 3-hydroxy-2-methylvaleric acid and 3-hydroxy-2,6-dimethyl-5-heptenoic acid.

17. The method of claim 15, wherein the hydroxyalkanoate is at least one selected from the group consisting of 3-hydroxybutyrate, 3-hydroxyvalerate, 4-hydroxybutyrate, (D)-3-hydroxycarboxylic acids with a medium chain length of 6 to 14 carbon numbers, 3-hydroxypropionic acid, 3-hydroxyhexanic acid, 3-hydroxyheptanoic acid, 3-hydroxyoctanoic acid, 3-hydroxynonanoic acid, 3-hydroxydecanoic acid, 3-hydroxyundecanoic acid, 3-hydroxydodecanoic acid, 3-hydroxytetradecanoic acid, 3-hydroxyhexadecanoic acid, 4-hydroxyvaleric acid, 4-hydroxyhexanoic acid, 4-hydroxyheptanoic acid, 4-hydroxyoctanoic acid, 4-hydroxydecanoic acid, 5-hydroxyvaleric acid, 5-hydroxyhexanoic acid, 6-hydroxydodecanoic acid, 3-hydroxy-4-pentenoic acid, 3-hydroxy-4-trans-hexenoic acid, 3-hydroxy-4-cis-hexenoic acid, 3-hydroxy-5-hyxenoic acid, 3-hydroxy-6-trans-octenoic acid, 3-hydroxy-6-cis octenoic acid, 3-hydroxy-7-octenoic acid, 3-hydroxy-8-nonenoic acid, 3-hydroxy-9-decenoic acid, 3-hydroxy-5-cis-dodecenoic acid, 3-hydroxy-5-cis-dodecenoic acid, 3-hydroxy-5-cis-tetradecenoic acid, 3-hydroxy-7-cis-tetradecenoic acid, 3-hydroxy-5,8-cis-cis-tetradecenoic acid, 3-hydroxy-4-methylvaleric acid, 3-hydroxy-4-methylhexanoic acid, 3-hydroxy-5-methylhexanoic acid, 3-hydroxy-6-methylhexanoic acid, 3-hydroxy-4-methyloctanoic acid, 3-hydroxy-5-methyloctanoic acid, 3-hydroxy-6-methyloctanoic acid, 3-hydroxy-7-methyloctanoic acid, 3-hydroxy-6-methylnonanoic acid, 3-hydroxy-7-methylnonanoic acid, 3-hydroxy-8-methylnonanoic acid, 3-hydroxy-7-methyldecanoic acid, 3-hydroxy-9-methyldecanoic acid, 3-hydroxy-7-methyl-6-octenoic acid, malic acid, 3-hydroxysuccinic acid-methylester, 3-hydroxyadipinic acid-methylester, 3-hydroxysuberic acid-methylester, 3-hydroxyazelaic acid-methylester, 3-hydroxysebacic acid-methylester, 3-hydroxysuberic acid-ethylester, 3-hydroxysebacic acid-ethylester, 3-hydroxypimelic acid-propylester, 3-hydroxysebacic acid-benzylester, 3-hydroxy-8-acetoxyoctanoic acid, 3-hydroxy-9-acetoxynonanoic acid, phenoxy-3-hydroxybutyric acid, phenoxy-3-hydroxyvaleric acid, phenoxy-3-hydroxyheptanoic acid, phenoxy-3-hydroxyoctanoic acid, para-cyanophenoxy-3-hydroxybutyric acid, para-cyanophenoxy-3-hydroxyvaleric acid, para-cyanophenoxy-3-hydroxyhexanoic acid, para-nitrophenoxy-3-hydroxyhexanoic acid, 3-hydroxy-5-phenylvaleric acid, 3-hydroxy-5-cyclohexylbutyric acid, 3,12-dihydroxydodecanoic acid, 3,8-dihydroxy-5-cis-tetradecenoic acid, 3-hydroxy-4,5-epoxydecanoic acid, 3-hydroxy-6,7-epoxydodecanoic acid, 3-hydroxy-8,9-epoxy-5,6-cis-tetradecanoic acid, 7-cyano-3-hydroxyheptanoic acid, 7-cyano-3-hydroxyheptanoic acid, 3-hydroxy-7-fluoroheptanoic acid, 3-hydroxy-9-fluorononanoic acid, 3-hydroxy-6-chlorohexanoic acid, 3-hydroxy-8-chlorooctanoic acid, 3-hydroxy-6-bromohexanoic acid, 3-hydroxy-8-bromooctanoic acid, 3-hydroxy-11-bromoundecanoic acid, 3-hydroxy-2-butenoic acid, 6-hydroxy-3-dodecenoic acid, 3-hydroxy-2-methylbutyric acid, 3-hydroxy-2-methylvaleric acid and 3-hydroxy-2,6-dimethyl-5-heptenoic acid.
